(19) United States
(12) Patent Application Publication
(10) Pub. No.: US 2026/0069255 A1
(43) Pub. Date: Mar. 12, 2026

(54) SYSTEMS AND METHODS FOR ENHANCED ECHOCARDIOGRAPHY FOR CARDIOVASCULAR DISEASE DETECTION

(71) Applicant: Steven C. Smart, St. Louis, MO (US)

(72) Inventor: Steven C. Smart, St. Louis, MO (US)

(21) Appl. No.: 19/321,998

(22) Filed: Sep. 8, 2025

Related U.S. Application Data
(60) Provisional application No. 63/692,358, filed on Sep. 9, 2024.

(51) Int. Cl. A61B 8/00; A61B 8/06; A61B 8/08; G06T 7/00; G16H 10/60; G16H 30/40; G16H 40/63; G16H 50/20

(52) U.S. Cl. CPC A61B 8/58; A61B 8/06; A61B 8/0883; A61B 8/488; A61B 8/5215; G06T 7/0012; G16H 10/60; G16H 30/40; G16H 40/63; G16H 50/20; G06T 2207/20081; G06T 2207/30048; G06T 2207/30104

(57) ABSTRACT

A smart echocardiography (ECHO) system includes a processor programmed to access a two-stage machine-learning (ML) model for analyzing echocardiograms. The two-stage ML model is trained to output a validated cardiac profile of a patient having improved accuracy based upon an inputted echocardiogram. The processor is further programmed to receive echocardiographic imaging data of a patient from the inputted echocardiogram and execute a first-stage of the two-stage ML model to generate an initial cardiac profile based on the echocardiographic imaging data. The initial cardiac profile includes a plurality of cardiac parameters each having a parameter value. The processor is further programmed to execute a second stage of the two-stage ML model on the initial cardiac profile by executing a plurality of validation calculations using the plurality of cardiac parameters and associated parameter values to generate a validated cardiac profile for the patient and output the validated cardiac profile.

### CROSS-REFERENCE TO RELATED APPLICATION

This application claims priority to U.S. Provisional Application No. 63/692,358, filed Sep. 9, 2024, the entire contents of which are incorporated herein by reference.

### FIELD OF THE DISCLOSURE

The field of the disclosure relates generally to echocardiography (ECHO) for detecting cardiovascular disease (CVD), and more specifically, to advanced echocardiographic techniques for enhanced CVD imaging.

### BACKGROUND

Cardiovascular disease (CVD) is optimally detected during phases in which it is most preventable, treatable, dynamic, and accelerating in progression. Early and accurate identification of such stages enables timely intervention, improved patient outcomes, and reduced healthcare burden. Echocardiography is an imaging technology for diagnosis, management and prognostication of cardiac disease. During an echocardiography, a probe, such as a transducer, is placed on a chest of a patient over the patient's heart. The probe emits ultrasound waves that bounce off the heart structures and return as echoes. These echoes are then converted into images on a display monitor.

Echocardiography oftentimes depends on the expertise of sonographers to correctly obtain images and both sonographers and echocardiographers to extract structure/function of the heart. However, the ECHO methods in use today have evolved to a level of sophistication beyond the abilities of most sonographers and echocardiographers to manually process. This has caused frustration with many quality and workflow issues in imaging and echocardiography. Timely best practice echocardiography is no longer feasible without a large team of highly trained/experienced imaging and analytic sonographers and providers/echocardiographers. Health care demands greater productivity, highest quality, quantitative analytics, and immediate reporting in the area of ECHO, but major limiting factors including time, inexperience, high volumes, and economics, need to be addressed.

Accordingly, it would be desirable to have a system that provides improved consistency and accuracy in the practice of echocardiography.

### BRIEF DESCRIPTION

In one aspect, a smart ECHO system is provided. The smart ECHO system includes a computer system comprising at least one processor in communication with at least one memory. The at least one processor is programmed to access a two-stage ML model for analyzing echocardiograms. The two-stage ML model is trained to output a validated cardiac profile of a patient having improved accuracy based upon an inputted echocardiogram. The processor is further programmed to receive echocardiographic imaging data of a patient from the inputted echocardiogram and execute a first-stage of the two-stage ML model to generate an initial cardiac profile based on the echocardiographic imaging data. The initial cardiac profile includes a plurality of cardiac parameters each having a parameter value. The processor is further programmed to execute a second stage of the two-stage ML model on the initial cardiac profile by executing a plurality of validation calculations using the plurality of cardiac parameters and associated parameter values to generate a validated cardiac profile for the patient and output the validated cardiac profile.

In another aspect, a non-transitory computer readable medium including instructions stored thereon is provided. The instructions, when executed by at least one processor, cause the at least one processor to access a two-stage machine-learning (ML) model for analyzing echocardiograms. The two-stage ML model is trained to output a validated cardiac profile of a patient having improved accuracy based upon an inputted echocardiogram. The instructions further cause the at least one processor to receive echocardiographic imaging data of a patient from the inputted echocardiogram and execute a first-stage of the two-stage ML model to generate an initial cardiac profile based on the echocardiographic imaging data. The initial cardiac profile including a plurality of cardiac parameters each having a parameter value. The instructions further cause the at least one processor to execute a second stage of the two-stage ML model on the initial cardiac profile including executing a plurality of validation calculations using the plurality of cardiac parameters and associated parameter values to generate a validated cardiac profile for the patient. The instructions further cause the at least one processor to output the validated cardiac profile.

In yet another aspect, a computer-implemented method for analyzing echocardiography (ECHO) results is provided. The method includes accessing, from at least one memory, a two-stage machine-learning (ML) model for analyzing echocardiograms. The two-stage ML model is trained to output a validated cardiac profile of a patient having improved accuracy based upon an inputted echocardiogram. The method further includes receiving echocardiographic imaging data of a patient from the inputted echocardiogram and executing, by at least one processor in communication with the at least one memory, a first-stage of the two-stage ML model to generate an initial cardiac profile based on the echocardiographic imaging data. The initial cardiac profile includes a plurality of cardiac parameters each having a parameter value. The method further includes executing, by the at least one processor, a second stage of the two-stage ML model on the initial cardiac profile including executing a plurality of validation calculations using the plurality of cardiac parameters and associated parameter values to generate a validated cardiac profile for the patient. The method further includes outputting, by the at least one processor, the validated cardiac profile.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings

FIG. 1 illustrates an exemplary computer system for intelligent analysis of ECHO results.

FIG. 2 is a flow chart of a process for analyzing ECHO results using the system shown in FIG. 1.

FIG. 3 depicts an exemplary configuration of a user computer device in accordance with one embodiment of the present disclosure.

FIG. 4 depicts an exemplary configuration of a server computing device in accordance with one embodiment of the present disclosure.

Unless otherwise indicated, the drawings provided herein are meant to illustrate features of embodiments of this disclosure. These features are believed to be applicable in a wide variety of systems including one or more embodiments of this disclosure. As such, the drawings are not meant to include all conventional features known by those of ordinary skill in the art to be required for the practice of the embodiments disclosed herein.

### DETAILED DESCRIPTION

The field of the disclosure relates generally to echocardiography for detecting cardiovascular disease (CVD), and more specifically, to advanced echocardiographic techniques for enhanced CVD imaging. Furthermore, the systems and methods described herein recite a system for echocardiography using automation and refined constantly updated algorithms that use artificial intelligence.

ECHO/imaging must be as accurate for stability as for disease regression/progression. Providing proper health care includes accurate definition of mechanisms of decompensation. Accurate comparative analytics includes standardized analytics for all imaging studies and modalities. Proper healthcare demands not only real-time reporting but also accurate pathophysiology and magnitude and rate of change/complexity.

The systems and methods described herein recite a smart ECHO system being executed by one or more smart ECHO computer devices. The smart ECHO system automates imaging best practices and complete image and data analytics for accurate structural, physiologic, hemodynamic grading of cardiovascular disease and normalcy. The smart ECHO system 100 provides standardized image recognition and analytics and data analytics to reproducibly extract that unique cardiovascular profile with disease grading, complexity and magnitude and rates of change. The smart ECHO system provides a standardized automated validated algorithm for ECHO image acquisition and data analytics/reporting.

The human heart functions as an efficient physiologic pump under normal conditions but may become compromised in multiple ways when diseased. Effective imaging modalities should be capable of accurately grading and differentiating among disease states and quantifying disease severity, complexity, and extent, in order to enable appropriate diagnosis, staging, and therapeutic decision-making. Pumping of blood or circulation through the lungs and body is the primary heart function and definable by a profile of mechanics, flow dynamics, structure, strain, physics and mathematics. Echocardiography is the most common imaging for cardiovascular disease but may, in some instances, fail to most accurately measure the cardiac profile due to unrealistic time constraints and human error or lack of knowledge issues. ECHO lacks standardization so ultrasonic imaging, analytics and reporting are variable.

The systems and methods described herein utilize an algorithm of correct echocardiographic images, measurements and analytics of structure and function to facilitate data driven reporting by matrix mathematics, matrices of structure and function and physics. Correct imaging and analytics according to at least some known systems and methods often includes manual measurements and/or calculations, taking hours to days to complete with a usual team of a sonographer and expert echocardiographer. Such systems and methods are not well suited to achieving optimized clinical impact from ECHO results, such as real-time results reporting.

The algorithm of the systems and methods described herein may be based, at least partially, on supervised machine learning of a training set of manually annotated ECHOs. The training set may include 6,000-10,000 ECHOs manually annotated by one or more expert echocardiographers. The algorithm may be automated according to at least one of: i) a complete imaging protocol; ii) intelligent image and data analytics; iii) physics and matrix mathematics basis for data driven reporting; iv) coding of intelligent grading of disease severity and complexity. Evolve comparative and dimensional analytics for best cardiac profiling and disease trends, rates of change, prognosis.

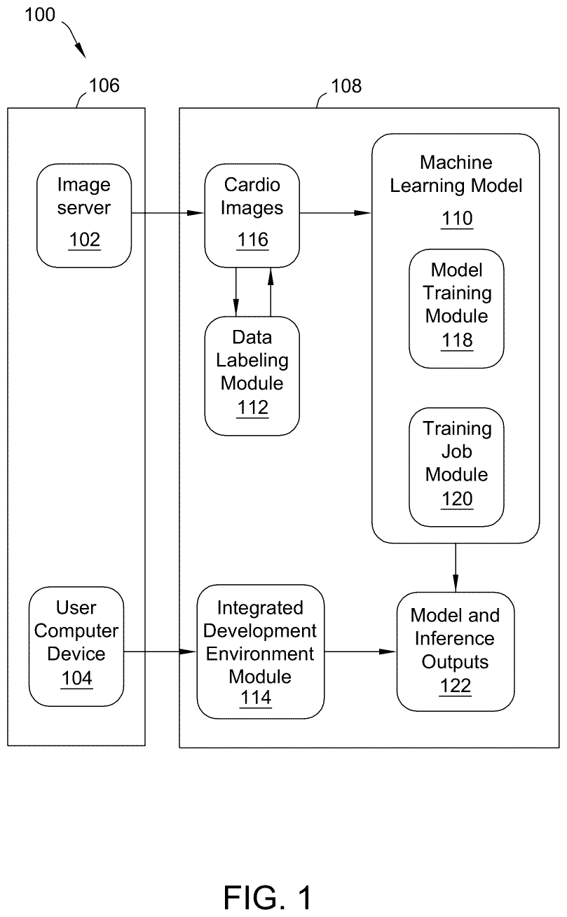

FIG. 1 shows an exemplary computer system 100, also referred to herein as the “smart ECHO system” or “smart ECHO”, for intelligent analysis of ECHO results.

Referring to FIG. 1, in the example embodiment, the computer system 100 includes an image server 102 and a user computer device 104, also referred to herein as a “user computer”. The user computer 104 may include a general purpose computing device, such as a client computer and/or mobile device. In the example embodiment, the image server 102 and the user computer 104 are “on-premise”, in that they are located at a medical site 106 where the ECHOs may be administered. The medical site 106 may include a hospital, doctor's office, and/or any other suitable medical treatment facility. The image server 102 and user computer 104 are each in communication with an external server system 108 located “off premise”. In some embodiments, the external server system 108 may include one or more remote cloud computing servers that collectively perform the functions as described herein with respect to the external server system 108.

In the example embodiment, the image server 102 is configured to store and manage ECHO results, including echocardiographic imaging data (also referred to herein as “imaging data”). In some embodiments, the image server 102 includes a Picture Archiving and Communications System (PACS) and/or a Vendor Neutral Archive (VNA). In the exemplary embodiment, the image server 102 is in communication with the external server system 108. The image server 102 is configured to transmit the echocardiographic imaging data to the external servers 108 for analysis according to the machine learning (ML) model 110, as described herein. In some embodiments the image server 102, and optionally the Picture Archiving and Communication System (PACS) of the image server 102 performs an initial analysis of the ECHO results prior to transmitting the ECHO results to the server system 108. For example, in some embodiments, the PACS analyzes the ECHO data, derives a report with the structural, physiologic and disease profile, and provides any feedback for completeness, image and data accuracy, gaps/missing data images, and need for higher-level analytics (structural or functional/pathophysiologic).

In the exemplary embodiment, the external server system 108 includes a data labeling module 112, the machine learning (ML) model 110, and an integrated development environment (IDE) module 114. The data labeling module 112, the machine learning (ML) model 110, and the IDE module 114 may include complimentary software modules or applications of an ML model service, such as a ML model development web service.

In the exemplary embodiment, the IDE module 114 includes a software development application that provides a graphical interface for the development and refinement of the ML model 110 (e.g., writing, debugging, testing, and/or updating software code). In some embodiments, the user computer device 104 may directly access other modules/outputs of the ML model 110, such as to view outputs, review changes to cardiac parameters, view reports from ML model 110, etc.

The data labeling module 112 is configured to perform labelling of complementary cardio images 116 (i.e., imaging data) for appropriate calculations/secondary/tertiary/quaternary data analytics. In some embodiments, the data labeling module 112 cooperates with the machine learning model, such that initial imaging data is labeled, analyzed by the ML model 110, and reanalyzed and/or relabeled by the data labeling module 112 (e.g., during the first or second stages).

The ML model 110 is configured to analyze echocardiograms information received from an echocardiogram, such as echocardiographic imaging data of a patient from an inputted echocardiogram. The ML model 110 includes a model training module 118 that is configured to train the ML model 110 to perform the calculations and operations, and generate the ECHO outputs, as described herein. For example, in the exemplary embodiment, the ML model 110 is trained based on database of principles and methods derived from training based on historical echocardiogram data. The historical echocardiogram data may include a plurality of historical echocardiographic imaging data and associated validated cardiac profiles. For example, the historical echocardiogram data may include a subset of prior echocardiograms that have been manually annotated (i.e., validated) by a sonographer/echocardiographer according to the procedures and principles described herein. A training job module 120 is configured to update the ML model 110 based on a new training job (e.g., as initiated by a user at user computer device 104).

In the example embodiment, the ML model 110 is configured to output a validated cardiac profile having improved accuracy based on the inputted echocardiogram. In some embodiments, the ML model 110 is a “two-stage ML model”, in that the analysis performed by the ML model 110 is performed in at least two stages, with the outputs from the initial or first stage being used as inputs for the second stage (e.g., in a feedback loop). While described as a “two-stage” ML model 110 herein, it should be understood that, in other embodiments, ML model 110 may incorporate more than two stages.

In the exemplary embodiment, the ML model 110 is configured to receive echocardiographic imaging data 116 of a patient from the inputted echocardiogram. Based on the received imaging data 116, the ML model 110 executes a first-stage of the two-stage ML model to generate an initial cardiac profile.

The initial cardiac profile may include a plurality of cardiac parameters each having a parameter value. The plurality of cardiac parameters may each represent at least one of a structural and functional feature of a heart of the patient and the parameter values represent a condition of the at least one of the structural and functional feature. Example cardiac parameters may include, but are not limited to, the following parameters: i) blood flow direction along a sample line of flow, ii) blood flow velocity along the sample line of flow, iii) aortic and pulmonary valve stroke volume calculation, iv) mitral and tricuspid valve stroke volume calculation, v) shunt volume, and vi) transvalvular total net stroke volume. In some embodiments, the parameter values of the plurality of cardiac parameters are determined based on at least one of image measurements from the echocardiographic imaging data and calculations performed based on the image measurements. For example, the parameter values may include initial calculations of lengths, areas, or volumes of one or more regions of the heart that are generated based on an image analysis of the inputted echocardiogram.

The ML model 110 is further configured to execute a second stage of the ML model 110 on the initial cardiac profile. Execution of the second stage of the ML model 110 may include executing a plurality of validation calculations using the plurality of cardiac parameters and associated parameter values to generate model inferences and outputs, indicated at module 122. The inferences and outputs 122 may include a validated cardiac profile for the patient. The model 110 may output the validated cardiac profile to the user computer device 104. In some embodiments, the validating of the second stage of the ML model 110 includes changing one or more of the initial parameter values of the initial cardiac profile. The changes to the initial parameter values may result from execution of the plurality of validation calculations and/or one or more inferences generated by the second stage of the ML model 110 based on the initial cardiac profile, when analyzed by the ML model 110 in its entirety. The validation calculations and inferences may be based on certain principles and/or rules, as described herein, related to measurements of the heart. The validated cardiac profile may include at least one of a structural, functional, and disease profile of a heart of the patient

In some embodiments, the smart ECHO system 100 is programmed to determine a diagnosis for the patient based on the validated cardiac profile and/or generate a report based on the validated cardiac profile.

In some embodiments, the smart ECHO system 100 is configured to perform a real-time dimensional analysis of one or more cardiac parameters, such as blood flow direction and change in blood flow rate as a function of time.

In the example embodiment, the smart ECHO system 100 is programmed to include the following principles, such as, but not limited to: structure, phasic mobility, phasic flow, backscatter, video density, texture, amplitude, temporal resolution, fundamental imaging, tissue and contrast harmonics, filters, compression, time gain, pulsed wave Doppler, continuous wave Doppler, m-mode/2D/3D/4D imaging and Doppler, frequency, spatial resolution, algebra, geometry, trigonometry, calculus, matrix mathematics, conservation of flow/mass and the physics of gradients, laminar flow, turbulence, pressure recovery, artificial and congenital structures, acquired and degenerative changes, resistance, impedance, radial/longitudinal/tortional strain, strain rate, motion in space, acceleration, deceleration, velocity, displacement, turbulence, flow, rates of change, volume, normalization (height, mass, BSA, and Z scores), venous pressures, chamber pressures, vascular pressures, central arterial and peripheral pressures/flow in extremities. The structure validates physiology and flow dynamics. The physiology and flow dynamics in turn validate structure. Global data validates the regional data and the reverse.

The smart ECHO system 100 uses supervised machine learning to automate and refine methods of image and data analytics. Using the results of the machine learning, the smart ECHO system 100 derives the correct structural, functional and disease profile of each patient and works to improve its derivation. In addition to best practice image analytics, the smart ECHO system 100 also uses archived extractable image data (video density, texture, low velocity motion, backscatter, tissue and density borders, incidental findings apparent to human vision and beyond that of human vision). By using the correctly derived structural, functional and disease profiles, the smart ECHO system 100 is programmed for unsupervised machine learning, transfer learning, deep learning and neural network development, and finally natural learning to learn newer, faster, more accurate and efficient ways to derive the correct structural, physiologic and disease profiles for the patients.

Furthermore, each time the dataset of studies reaches preset or learned thresholds of size, the smart ECHO system 100 re-executes and refines analytics to screen for new and improved analytics. At set intervals, the smart ECHO system 100 also accesses websites associated with societies and literature for major new developments and incorporates those new developments as appropriate to provide improved up-to date best image practice, comparative, and data analytics.

The smart ECHO system 100 uploads the studies into the system before completion of the exam. In some embodiments, the smart ECHO system 100 can be incorporated with a Picture Archiving and Communication System (PACS) to analyze the data, derive a report with the structural, physiologic and disease profile and provide any feedback for completeness, image and data accuracy, gaps/missing data images, and need for higher-level analytics (structural or functional/pathophysiologic). Any supplemental imaging is done and the smart ECHO system 100 re-executes the analytics to detect normalcy or the correct disease profile. In at least some embodiments, the smart ECHO system 100 may also analyze historical legacy studies to standardize and automate comparative analytics.

In the exemplary embodiment, the smart ECHO system 100 is programmed to implement the following principles, such as, but not limited to, in generating an initial cardiac profile and/or forming as a basis for the plurality of validation calculations:
1. Conservation of mass—the total venous return/inflow always equals aortic outflow (basal cardiac stroke volume).
2. The proximal isovelocity surface area (PISA) method is used for orifice area (hemisphere, hemicylinder, semi-hemicylinder and multiple corrections/modifications) and shunt or regurgitant volume is done with color Doppler Nyquist limit (NL) set for ˜60±5 cm/sec and then baseline shifting toward the receiving chamber to NL of 35-45 cm/sec (best shape of isovelocity shells for formulas).
3. The aortic and pulmonic valve systolic stroke volumes equal the basal cardiac stroke volume plus the regurgitant volume of AR (aortic valve regurgitation) or PR (pulmonic valve regurgitation), respectively. If the valves are part of a shunt circuit, the valve stroke volume also includes the shunt volume.
4. The mitral and tricuspid valve stroke volumes equal basal cardiac stroke volume plus regurgitant volume of MR (mitral regurgitation) or TR (tricuspid regurgitation), respectively. If the valves are part of a shunt circuit, the valve stroke volume also includes the shunt volume.
5. The left ventricular stroke volume equals basal cardiac stroke volume plus mitral regurgitant volume, aortic regurgitant volume, and shunt volume if involved in a shunt circuit.
6. The two dimensional (2D) and three dimensional (3D) left ventricular stroke volume and ejection fraction are validated by the 2D/3D Doppler data. The reverse also true.
7. The Ejection fraction (EF) is validated by the 2D and 3D Doppler and valvular stroke volume data.
8. The basal cardiac stroke volume equals the transaortic or trans-pulmonic valve flow when there is no valve regurgitation.
9. The right ventricular stroke volume equals the basal cardiac stroke volume plus the tricuspid regurgitant volume, the pulmonic regurgitant volume and shunt volume if involved in shunt circuit.
10. The effective regurgitant orifice area (EROA) and volume (RV) by the PISA (proximal isovelocity surface area) method when done correctly equal the RV and EROA by the valvar stroke volume method (conservation of mass). The smart ECHO system 100 uses the hemisphere formula when there is a single hemispheric jet of regurgitation. The smart ECHO system 100 uses the multiple hemisphere formula when there are multiple hemispheric jets. The smart ECHO system 100 uses the hemicylinder formula when there is wide origin/elongated shape. The smart ECHO system 100 used the semi-hemicylinder when the EROA is a non-linear shape. And the smart ECHO uses multiples and/or combinations of the above when appropriate.
11. 2D/3D left and right ventricular stroke volumes area corroborated by the sum of the basal cardiac stroke volume, semilunar valve regurgitant volume, atrioventricular (AV) valve regurgitant volume and shunt volume if involved in shunt circuit.
12. EROA is derived from regurgitant volume by quotient of regurgitant volume and velocity time integral (VTI) of the regurgitant flow by continuous wave (CW) Doppler and equals the PISA derived regurgitant orifice area by correct analytics.
13. Regurgitant fraction is the quotient of regurgitant volume and the transvalvular stroke volume cross validated by PISA and volume methods.
14. Basal cardiac flow equals the difference of transvalvular flow and regurgitant volume as derived by correct PISA analytics and volume method analytics.
15. Regurgitant fraction is derived by the quotient of the regurgitant volume and transvalvular stroke volume for each respective valve.
16. Regurgitant volume and fraction are also derived from the left ventricular (2D and 3D) and right ventricular (3D data sets) volume data and the transvalvular stroke volumes especially alone when there is a valve with minimal or no regurgitation and no shunt. Analytics are also valid complex disease by the use PISA analytics when all valve leak and/or shunt present.
17. Regurgitant volume equals the difference of transvalvular stroke volume or LV stroke volume and basal cardiac stroke volume/transvalvular stroke volume of any valve with no leak. Regurgitant fraction by the same formula and is validated by correct PISA analytics.
18. Shunts are corroborated by PISA analytics of color flow and spectral Doppler data from the sending to the receiving chamber and chamber and valve stroke volume data.
19. Shunt volume by PISA method with correct analytics equals the shunt volume based on differences in valvular stroke volume data and LV/RV stroke volume data. The smart ECHO system 100 uses the hemisphere formula when there is a single hemispheric jet of regurgitation. The smart ECHO system 100 uses the multiple hemisphere formula when there are multiple hemispheric jets. The smart ECHO system 100 uses the hemicylinder formula when there is wide origin/elongated shape. The smart ECHO system 100 used the semi-hemicylinder when the EROA is a non-linear shape. And the smart ECHO uses multiples and/or combinations of the above when appropriate.
20. When complex valve disease and regurgitation are detected, shunt volumes and Qp/Qs are calculated by subtraction of regurgitant volumes from chamber stroke volumes and transvalvular stroke volumes to reflect only basal cardiac stroke volume and shunt volume.
21. 2D and 3D planimetry measured valve area equals valve area calculated by continuity equation (conservation of mass) cross validated by the volume data (chamber and transvalvular stroke volumes) and also correlates with m-mode excursion for semilunar valves (pulmonic and aortic) except for asymmetric orifii. Enhanced automated analytics enable systolic PISA analytics for semilunar valve area with angle correction for constraint as appropriate.
22. Continuous Wave (CW) Doppler within the heart reflects flow across the valve (smallest area of flow in the flow circuit) except when there is worse sub-valvular or supra-valvular stenosis/obstruction. CW Doppler reflects mitral and tricuspid inflow gradients and flow/diastolic function. Pulsed Wave (PW) Doppler at leaflet tips is used when AR or PR preclude use of CW Doppler data. PW Doppler at annulus (correct positioning is at least one relevant factor) and area (shape analytics being at least one relevant factor, biplane 2D diameters and 2D and 3D planimetry/area) calculate transvalvular stroke volume.
23. Continuity equation for peak and mean velocities and VTIs is validated and certified by conservation of mass (flow and stroke volume must be the same for the sites of PW and CW data collection). The quotient of trans valvar stroke volume and CW Doppler VTI calculates maximum valve orifice area when opening is monophasic (semilunar valves) and mean orifice area when opening is multiphasic (mitral and tricuspid AV valves).
24. Peak velocity PW/CW ratios reflect maximum valve area while mean velocity and VTI ratios reflect mean valve area (new concept) with phasic flow and multiphasic valve opening (mitral and tricuspid AV valves and are corroborated by maximum m-mode, 2D, 3D and 4D imaging and mean m-mode, 2D, 3D and 4D imaging at 20 msec intervals when automation of image analytics is perfected). AV and PV peak (Peak pressure 4(Vav)²) and mean gradients (VTI of CW Doppler of AV) are assessed by simple Bernoulli equation in normal flow states with normal to low flow and normal to large aortas/pulmonary arteries. The modified Bernoulli equation (4[(Vav)²−(Vlvot)²]) is used for small/obstructed Left Ventricular Outflow Tract (LVOT) and/or Right Ventricular Outflow Tract (RVOT) and high flow states (LVOT velocity>1.5 m/sec) and small aortas/pulmonary arteries (ST junction/proximal aorta<3.0 cm, main PA<2.5 cm). In cases of small valves and high turbulence/prosthetics with multiple orifii, the pressure recovery correction {4(Vav-peak or mean)²×2×AVA/AA×(1−AVA/AA)} adjusts for correct peak and mean gradients. The central orifice of triple orifice mechanical valves peak/mean velocities and VTI are determined but clinical gradients are derived by disc orifii peak/mean velocities and VTI with modified Bernoulli equation and pressure recovery correction as needed. Area and flow calculations area derived from the velocity (A1×V1=A2×V2) and VTI (A1×VTI1=A2×VTI2) based continuity equations and conservation of mass principle with corroboration by direct 3D/4D planimetry (maximum for now and mean when automation of image analytics perfected). The smart ECHO uses machine learning and deep learning to refine and derive best algorithm.
25. Conservation of mass (flow and stroke volume are the same for the sites of PW and CW data collection) and HPRF PW Doppler is utilized when there is any degree of sub-valvular or supra-valvular stenosis and/or high flow states.
26. Areas at any Doppler site in the conserved flow circuit are derived by conservation of mass (VTI) and the continuity equation/conservation of mass. Valve area is derived by continuity equation correlates with planimetry valve area even in the setting of fixed/dynamic sub-valvular or supravalvular obstruction (minimum area by 2D or 3D planimetry at site of obstruction).
27. Delayed timing of peak velocity (scimitar sign) or multiple peaks reflect dynamic obstruction and multiphasic flow reflects dynamic obstruction and reopening.
28. PISA for valve orifice area (diastole for MV and TV, systole for AV and PV) again necessitate careful shape analysis and the additional parameter of valve constraint of flow (mean angle of constraint (θ) with the additional term of θ/180). Trigonometry will be considered for machine learning and further algorithm refinement as appropriate. The smart ECHO system 100 uses the hemisphere formula when there is a single orifice and hemispheric flow. The smart ECHO system 100 uses the multiple hemisphere formula when there are multiple hemisphere formula when multiple orifii and hemispheric flow. The smart ECHO system 100 uses the hemicylinder formula when there is a linear shape and hemicylinder linear flow. The smart ECHO system 100 used the semi-hemicylinder when there is a non-linear shape and nonlinear semi-hemicylinder flow. And the smart ECHO uses multiples and/or combinations of the above when appropriate.
29. Peak and mean velocities/VTI and PISA analysis produce the similar areas when flow is monophasic/parabolic. Area correlates with and is corroborated with by maximum area by 2D/3D planimetry and m-mode for circular valves.
30. For valves with multiphasic opening (mitral and tricuspid), VTI/mean velocities reflect mean orifice area and correlate with PISA analysis for early and late peaks (new concepts). Maximum area by correct PISA analytics correlates with and is corroborated by pressure halftime data and maximum area by 2D/3D planimetry.
31. The additional term of valve VTI is used to validate mean size data by conservation of mass (new concept).
32. PISA Doppler analytics and volume analytics are as accurate for small regurgitant lesions and shunts as well as medium to large regurgitant lesions and shunts.
33. M-mode correlates with 2D and 3D measurements validate each other when correctly imaged, captured and measured (especially circular valves and elliptical valves but not complex degenerative valves). The smart ECHO system 100 uses predictive analytics to anticipate valves of related measurements (conflict resolution feedback loop).
34. Structural information validates functional/Doppler data and the reverse when correctly imaged, captured and measured. Normal dimensions for all structures and values normalized to body size and height (chambers, walls, valves, membranes, septae, intra-cardiac, systemic vascular, pulmonary vascular and extra-cardiac structures) are reassessed at set intervals and re-derived as thresholds area reached for growth of the database/number of studies. Normal values and disease grades are intermittently adjusted for any significant changes as database size grows. All surgical, congenital, acquired, chamber, valvular, septal, shunt, systemic vascular, pulmonary vascular, intra-cardiac, membrane, and extra-cardiac pathologic structures are measured for m-mode, 2D, 3D and 4D dimensions (length, width, depth, excursion, area, volume, distance from and involvement of relevant adjacent structures). Data is auto-cross referenced/validated with Doppler data. The smart ECHO system 100 uses predictive analytics to anticipate valves of related measurements (conflict resolution servo feedback loop).
35. Pressure halftime principle according to standard diastolic inflow formula for area of AV valves, pressure equalization and severity of semilunar valve insufficiency.
36. The smart ECHO system 100 uses Artificial intelligence, unsupervised machine learning, neural network development/deep learning, and natural learning to invoke specialized and supplemental analytics for all APPROPRIATE lesions. Predictive analytics, identification of cause of errors and real time resolution of errors and conflicts are based on highest confidence of measurements (conflict resolution servo feedback loop).

In the exemplary embodiment, smart ECHO measurements, used for example in generating an initial cardiac profile and/or forming as a basis for the plurality of validation calculations, may include the following methods, techniques, and/or calculations:
1. Continuous wave (CW) Doppler measures direction and flow/velocities at all points along the sample line of flow with no limit to velocity.
2. Pulsed wave (PW) Doppler measures flow direction and velocities of flow or motion at the location of the sampling volume site (blood flow with low velocity filters and cardiac structures with high velocity filters).
3. CW and PW Doppler are accurate when parallel to 3D/4D flow.
4. Color Doppler is a plot/map of m-mode, 2D, 3D and 4D PW Doppler data.
5. High pulse repetition frequency (HPRF) pulsed wave Doppler is used when velocities are higher than the Nyquist limit to maintain detection of direction and velocity of flow (secondary sample volume data are filtered by the highest velocity occurring at the sampling site).
6. Aortic and pulmonary valve stroke volume calculation: LVOT (left ventricular outflow tract) or RVOT (right ventricular outflow tract) biplane/multiplane diameter calculated or 2D/3D measured area×LVOT or RVOT systolic VTI, respectively, at same site/timing as area/diameter measurement. Direct measured area to be gold standard.
7. Mitral and tricuspid valve stroke volume calculation: MV or TV annulus biplane/multiplane diameter calculated or 2D/3D measured area×MV annulus or TV annulus diastolic VTI, respectively, at same site as area/diameter measurement. Direct measured area to be gold standard.
8. In the absence of valve disease, shunt volume is the difference between LV (LVOT/transaortic) and RV (RVOT/trans-pulmonary) stroke volumes and Qp/Qs ratio derived from the same data.
9. Transvalvular total (including regurgitant volume) and net stroke (after subtraction of regurgitant volume) volumes indexed to body surface area (BSA, M2, height and lean body mass) reflect the cardiac index and flow/output status (low [<35 ml/M2/cycle, cardiac index<2.2 L/min/M2], normal and high [>110 ml/M2/cycle, cardiac index>5.5 L/min/M2]).
10. PW Doppler is done for AV and PV valves with the sampling volume just ventricular to the valve annulus in the LVOT and RVOT (shape analytics being at least one relevant factor, biplane 2D diameters and 2D and 3D planimetry/area).
11. Ratios of PW/CW for peak velocity, mean velocity and VTI are similar and reflect maximum valve orifice area with fixed monophasic opening (aortic and pulmonic semilunar valves and corroborated by m-mode, 2D, 3D and 4D imaging).
12. Area and PW/CW Doppler (velocities and VTIs) are measured at sites of obstruction and all sites in flow circuit (where flow is conserved, MV SAM/membrane/ridge sites, proximal to immediate sub-valvular LVOT/RVOT and sinuses of Valsalva/proximal pulmonary artery or higher if supravalvular obstruction). PW/CW Doppler is only measured without area at sites where flow is not conserved (peak and mean gradients, basal mid and apical LV/RV).
13. PW Doppler with baseline shift for maximized NL is used when there is no aliasing (velocities>NL and loss of direction of flow data) and HPRF PW when velocities are high and aliased.
14. CW Doppler maximum velocity and VTI reflect flow at site of worst obstruction.
15. For sub- or supra-valvular aortic obstruction, area (2D, X-plane and 3D/4D) and flow are measured at apical LV, mid LV, basal LV, site of obstruction and also at typical sites for LVOT near valve/1 cm ventricular to valve, at valve, in sinuses of Valsalva and above any obstruction in aorta.
16. For sub- or supra-valvular pulmonic obstruction, area and flow are measured at site of obstruction and also at typical sites for RVOT near valve/1 cm ventricular to valve, at valve, in proximal/mid/distal main/branch pulmonary artery and above any obstruction in pulmonary artery.
17. For sub- or supra-valvular mitral obstruction, area and flow are measured at site of obstruction and also at typical sites of annulus, 1 cm ventricular to tips of valve, at valve, and above any obstruction in LA.
18. For sub- or supra-valvular tricuspid obstruction, area and flow are measured at site of obstruction and at typical sites of annulus, 1 cm ventricular to tips of valve, valve, and above any obstruction in RA.
19. Early PR diastolic velocity/gradient correlates with mean TR gradient and mean PA pressure with the addition of RA/venous pressure (based on IVC/SVC and HV imaging/Doppler analytics). Standardized 0.61×peak TR gradient is used for corroboration. Late diastolic PR velocity/gradient correlates with PA diastolic pressure with the addition of RA/venous pressure (based on IVC/SVC and HV imaging/Doppler analytics). Pulmonary vascular resistance (PVR) is derived by Mayo formula in patients with normal PA pressure to mild pulmonary hypertension (TR velocity<3 m/sec) and the Schiller formula (5×(TRpeak (m/sec))²)/RVOT VTI (cm) with moderate to severe pulmonary hypertension (TR velocity>=3.5 m/sec). For moderate pulmonary hypertension (TR velocities of 3 to <3.5 m/sec), PVR is the mean of the two methods.
20. All stroke volume and chamber volumes are corrected for BSA/body size/height and cardiac anatomy and vascular structures normalized to Z scores, height, and area.
21. Enhanced analytics are invoked for any alterations in structure (e.g., pathologic LV and RV interdependence/discordance), structural relationships, and flow dynamics, based on pressures, flows, congenital anomalies, acquired anomalies, and systemic or pulmonary vascular alterations.

The smart ECHO system is comprehensive in scope to handle any cardiovascular condition/complexity and may have greatest impact in underserved areas by ensuring front end best practice imaging and analytics even in the absence of on-site best practice trained sonographers and echocardiographers. The smart ECHO system 100 automates and standardizes all measurements and calculations with matrix mathematics/physics and auto-validation. The smart ECHO system 100 provides near real-time feedback to the sonographer and echocardiographer. In some embodiments, the smart ECHO system 100 displays (e.g., at user computer 104 or any suitable computing device) implications of findings with disease profiles and complexity. The smart ECHO system 100 certifies proper comparative analytics to legacy ECHOs and other imaging, such as computed tomography (CT), magnetic resonance imaging (MRI), nuclear cardiology, positron emission tomography (PET)/single photon emission computed tomography (SPECT), and angiography and catheterization.

The smart ECHO system 100 executes an algorithm imbedded with AI, ML, and best practices to automated image acquisition, image/data analytics, and reporting of echocardiography for simultaneous best practice quality at improved workflow speeds.

An echocardiogram uses sound waves to produce images of a patient's heart. This common test permits real time assessment of cardiac structure and function. The healthcare provider can use the images from an echocardiogram to identify heart disease.

Depending on what information the healthcare provider needs, the patient may have one of several types of echocardiograms. The healthcare provider can suggest an echocardiogram to: a) check for problems with the valves or chambers of the heart; b) check if heart problems are the cause of symptoms such as shortness of breath or chest pain; c) fluid collections; d) major systemic or pulmonary vascular abnormalities; and e) abnormal or missing cardiac and systemic or pulmonary vascular connections.

A transthoracic echocardiogram is the standard type of echocardiogram. A technician (sonographer) spreads gel on a device (transducer). The sonographer presses the transducer firmly against the patient's skin, aiming an ultrasound beam through their chest to their heart. The transducer records the reflected sound wave echoes from the patient's heart. A computer converts the echoes into m-mode, 2D, 3D or 4D moving images on a monitor. If the patient's lungs or ribs block the view, the healthcare provider may need to provide a small amount of an enhancing agent (phospholipid/albumin gas filled microbubbles) injected through an intravenous (IV) line. The enhancing agent is safe, well tolerated and enhance the heart's structures, Doppler and blood flow for best practice analytics.

If the healthcare provider wants more-detailed images or it's difficult to get a clear picture of the patient's heart with a standard echocardiogram, the healthcare provider may use a transesophageal echocardiogram. The patient's throat is numbed, and is given medications to help the patient relax. A flexible tube containing a transducer is guided down the patient's throat and into the esophagus and the stomach. The transducer records the sound wave echoes from the patient's heart. A computer converts the echoes into detailed m-mode, 2D, 3D and 4D moving images of cardiac structure, function and pathology/pathophysiology.

In other cases, color flow and spectral Doppler is used to image high or low velocity blood flow the heart and blood vessels and low velocity tissue and valve motion. Speckle tracking can also track tissue, valve and blood flow. Direction, timing, duration, velocity, flow (integral of velocity over time), rates of change and acceleration, deceleration, duration of decay and duration of acceleration are relevant factors/useful parameters. Doppler techniques are essential and unique for all transthoracic and transesophageal echocardiograms especially in the assessment of normal/abnormal blood flow, valves, chambers, tissue motion/velocity/acceleration and pressure gradients. M-mode, 2D, 3D and 4D color flow Doppler can be used to enhance detection of normal/abnormal flow and quantify pathology.

Some heart problems, particularly those involving the arteries that supply blood to the heart muscle (i.e., coronary arteries/CAD) are only detectable during physical activity. The healthcare provider might use a stress echocardiogram to check for coronary artery problems/ischemia, diastolic dysfunction/stiffness, valve pathology, pulmonary vascular dysfunction and pulmonary hypertension. ECHO can only image the proximal left and right coronary arteries so is inadequate to screen for CAD.

In a stress echocardiogram, ultrasound images of the heart are taken before and immediately after the patient walks on a treadmill. If the patient is unable to exercise, the patient may be fused with a medication to simulate exercise. Limited or complete ECHO imaging is done also during exercise with supine bicycle stress and with pharmacologic stress. Real time perfusion imaging with contrast can also be done at rest and after stress to screen for CAD/infarction/types of myocardial injury.

The smart ECHO system 100 is configured to provide 1D/2D/3D/4D/multidimensional echocardiography to meet the current and future scientific, enhanced analytic, quality. and workflow demands. Modalities include adult resting echocardiogram, transesophageal echocardiogram (TEE), stress echocardiogram, and multidimensional stress/interventional echocardiograms.

Current community and academic standard for echocardiography is rapid performance and interpretation by qualitative or semiquantitative assessment. Some ECHO reports may be variable, not reproducible and/or not comparable due to conflicts and errors, failure to use best practice analytics, missing images, missing measurements, poor commitment to quality and analytic standards, invalidation of the laws of physics, excessive inter- and intra-observer variability, nonsensical data (regurgitant volumes>ventricular stroke volumes), abnormal data in normal echocardiograms, volumes incompatible with life or normal standards/healthy hemodynamic states, obstacles to quantitative analysis, and delayed or no feedback to technician performing the study.

Gaps in knowledge, exam protocols and the use of ever expanding and robust analytics cause readers and sonographers to discount the value and need for standards/analytics/mathematical and anatomic/functional inter-relationships. The high prevalence of gaps in data, gaps in analytics, gaps in quality, lack of awareness of the implications of measurements/analytics, erroneous data and conflicts lead readers and sonographers to discount and devalue quantitative analytics and focus only on workflow. Many/most reports are generated according to reader impression in spite of the measurements and analytics. Dimensional ECHOs such as stress ECHOs and interventional transesophageal (TEE) and transthoracic (TTE) echocardiograms almost never include quantitative analytics of function resulting in missed opportunity to describe/define relevant dimensional aspects of disease.

Groups/hospitals/systems, billing, accreditation, and administrative standards may suffer if improperly focused on study volume, workflow rather than data/interpretive standards and upstream/downstream quality. Known measure and methodologies for monitoring quality may be difficult to access or lacking in utility.

Current constraints on echocardiographers, readers, sonographers, and technicians include heightened productivity standards, limited access to productive tools, tight time pressures, reporting interval expectations, limited staffing, high expected volumes for sonographers, time limitations of exams/lack of access to off line measurement stations, limited communication of findings back to sonographers, limited feedback regarding quality, implementing uniform application of imaging best practices in view of changing standards, variable age of systems, variable logistics of machine use, variable display, variable key boarding, variable presets, variable setup controls, different setup controls, different capabilities of machines, variable expertise/commitment of sonographer/staff, laboratory size constraints, fewer studies in dedicated ECHO labs, more portable ECHOs, increased patient acuity and complexity, declining numbers of transporters, improper preparation of patients for studies, more conflicts with other studies and treatments, ongoing inconsistent reliability of transmission of data from machines to reading stations, ongoing network problems, ongoing issues with uploading of reports to HER/HER/communication to ordering providers and ever shorter intervals of exam times. The increased demand for speed of interpretation/qualitative impression may pose risks to reducing reliability and accuracy of the interpretations. Quality and outcome data are difficult to measure without intelligent analytic reports/intelligent tools. Appropriate use criteria/determination is not part of current ordering. ECHO volume/downstream test volume are the measures of growth. Complexity of studies, data quality, data interpretation, clinical utilization of data, impact and outcomes are not considered as growth/quality measures.

Embodiments of the present disclosure provide a system for conflict resolution with exams, historical data, symptoms, and other imaging. Differences and changes are performed upfront and able to be communicated in real-time. The systems of the present disclosure have access to industry standards, libraries of pathology and methods for best practice assessment and analytics. To make augmented intelligence a reality, systems of the present disclosure improve workflow though appropriate use of more limited exams and streamlined complex exams. Avoiding redundancy of methods/imaging, multitasking of methods, and multitasking of analytics may yield an augmented workflow framework and establish a novel domain of augmented workflow and intelligence.

The quality, completeness, correct analytics, and timeliness of imaging, according to the systems and methods of the present disclosure, are certified (e.g., by transparent methodologies) and guaranteed for all studies to support rather than confound the downstream machine learning and data analytics/data science that directly influences care and management. Imaging augmented intelligence and validation/certification analytics may push clinical algorithms for prevention, prognosis, and patient care, management, workflow, and logistics to the accuracy level needed to contain cost and significantly improve quality of life and outcomes (AUC (Area under the curve) by receiver operation characteristic (ROC) analysis/logistic/log loss analytics from 0.6-0.75 to the goal of >0.9 needed for confident decisions and care).

Technical improvements provided by the smart ECHO system include: minimized length of stay (LOS), reduced admissions and readmissions, maximal workflow/immediate reporting/communication, more accurate disease staging/extent/severity/complexity, reduced test redundancy, no unnecessary tests, universal best practice care, better prevention, better prognostication, risk reduction, improved quality of care, cost effectiveness, cost containment and improved quality of life and outcomes. Consistency of testing/evaluation procedures are improved as each ECHO is analyzed according to the same best practice protocol of predictive analytics, structural and hemodynamic profiling, and auto-validation according to the laws of physics, regression analytics and matrix mathematics. Consistency of testing/evaluation procedures are further improved by imbedding, constantly updating and streamlining use of best practices. Imbedding the essential laws of conservation of mass, the flow circuit map of the heart and the principles of flow dynamics/mechanics/mathematics in the acquisition/processing of ECHOs may provide for standardized studies/reports, guarantee completeness and accuracy of the primary anatomic and Doppler data, and ensure accuracy/reproducibility/comparability. Pretest predictive analytics optimize the use of limited imaging and complex investigative analytics.

At least some known ECHO systems incorporating some degree of automation are based on non-certified/non-validated measurements and calculations. Known systems are not based on data certified and validated by conservation of mass, Doppler/structure cross validation, flow dynamics, mathematics, matrix mathematics, and mechanics. Real-time modeling and feedback remain limited. Images are often not displayed in correct dimension. Visual modeling and display are not state of the art, so the implications, grade, and complexity of findings are not effectively communicated to the sonographers. Relevant missing data and gaps in data/findings are not communicated to the sonographer. Reports and displays are not formatted for structural, functional, and hemodynamic profiling of patients. Structural, functional, and hemodynamic profiles are not linked. The current analytic and reporting systems not only fail to recognize/flag conflicts but actually facilitate errors and conflicts by introduction of separate/nonoverlapping silos of data and information. Doppler has never been used to validate the volume/structural data. The result has been huge differences with other sophisticated imaging, gated MRI, and cine CT.

In the at least some known systems, conflicts due to the lack of built in matrices of interdependence have led to even less use of analytics for transesophageal, interventional, and stress studies. Predictive analytics are essential to data analytics and understanding how studies reproduce or deviate from normal stress responses and profiles. The issues and errors are magnified geometrically when data are collected without correct predictive and modeling analytics. Additionally, at least some known ECHO machines/systems imbed imaging methods/techniques but have limited or no intelligence. Protocols are tailored but not best practice or standardized. Studies depend on the training/expertise of the echocardiographer and sonographer. Feedback is limited or absent and conflicts not identified.

The smart ECHO system 100 described herein provides a technical solution to the above-described technical problems, for not only resting ECHO but also TEE, interventional and stress ECHO with best practice resting interdependent structural, functional, and hemodynamic profiling and historical comparative analytics and new innovative robust dimensional and predictive analytics for provoked (interventional and stress) and clinical status changes. The smart ECHO system 100 transforms ECHO into the hard science essential to achieve the goals of best practice prevention, screening, early detection, staging, prognosis, tailoring/selection/timing of interventions, and following evolution after intervention.

The smart ECHO system 100 utilizes best practice edge computing with a new front-end analytic add on module for the current ECHO machines and the network for sonographers and echocardiographers to immediately report and communicate results. The smart ECHO system 100 updates and imbeds the best practice protocols in the machines with a front-end feedback loop (e.g., the two stages of the two stage ML model 110) and dimensional/comparative analytics. The smart ECHO system 100 continuously links the sonographers, machines and echocardiographers. Time efficiency and productivity with be maximized for sonographers and echocardiographers with resolution of all bias in scheduling and time utilization.

The smart ECHO system 100 provides an improved means to benefit patient care by best practice disease severity, extent, stage, prevention, prognostication, disease stability/change/acceleration, workflow, communication, patient care, management and maximized clinical impact. Further benefits include simultaneous best practice quality, workflow, and communication. The 24/7 smart ECHO system 100 is configured to reduce barriers to the flow of data, data analytics, intelligence, information technology, logistics, prevention, prognosis, predictive analytics, image acquisition, time efficiency, image analysis, image dimensions, and inter-modality comparative, and dimensional analytics. Net effects of advances of the smart ECHO system 100 include reduced admissions, more timely and appropriate interventions, less inappropriate interventions, fewer complications, and shorter lengths of stay. The greater access irrespective of location reduces the differences in diagnostic standards related to economic, social, location and demographic differences. Seamless access to best practice imaging for all may be the new paradigm and key to universal standards, best practices, diagnostics, management, improved outcomes, quality of life, and economic productivity/education.

The interactive, display and analytic system with utilize edge computing and a new level of bidirectional feedback networking/connectivity with best practice and dimensionally correct display to remove the barriers to sonographer/echocardiographer awareness of findings, changes, and their implications. All nodes of the team may be aware of data analytics, feedback, communication, image display and logistics in real-time. Smart ECHO hardware and software/augmented intelligence can follow and improve on the role of 3D and Doppler and may finally find an imaging application of virtual reality and augmented reality image display/analysis/analytics/communication in cardiac and radiologic imaging.

Structural, functional, and hemodynamic profiling and anatomy/physiology specific processing of data with standardized historical comparative analytics to prior studies. The smart ECHO system 100 uses best practice historical and upfront analytics by ongoing deep learning, supervised machine learning and augmented intelligence. Furthermore, dimensional analytics ensure that all data are extracted with greater awareness of implications/clinical impact. Other downstream effects include immediate data feedback, enhanced awareness of changes and early identification of new issues, dysfunction, extent, severity, complexity, and changes in function. Clinical impact includes better utilization of interventions (a lesser need for other imaging), reduced inappropriate interventions, improved outcomes, and greater cost effectiveness/containment. The more complete investigation of disease process, elaboration of mechanisms and extent of disease, and staging of dysfunction directly impacts quality to accelerate patient return to compensation, recovery of quality of life, patient productivity, and enhanced primary and secondary prevention, including enhanced analysis of relevant incidental/associated findings.

Other direct effects of guaranteed accuracy of primary data, validation of related findings (resistance/pressure/impedance/strain/stress), data driven reporting, enhanced comparative analysis, better disease modeling, universal standardized reporting/best practice imaging, breakdown of location/demographic/training related barriers to imaging/data processing/analytics/time/workflow include simultaneous maximized workflow, speed, timeliness, quality, and analytic standards. Outside ECHOs also become immediately more useful by immediate standardized best practice analytics upon loading on the server. Studies analyzed according to inferior standards are no longer be a barrier. The smart ECHO system 100 correctly incorporates correct data into the dimensional profile of patients and better understand disease roles in presentations/clinical presentation irrespective of location.

Smart ECHO enhanced analytics based on vital sign changes, atrial fibrillation/flutter, atrial and ventricular arrhythmias permits the creation of structural/functional/hemodynamic profiles for all possible dimensions of disease (automated measurements/analytics at the same RR interval/same number of cycles/same hemodynamics to endure similar loading conditions). Predictive analytics for the benefits of interventions for heart rates, heart rhythms, blood pressure and systemic diseases with become newly available to clinicians for enhanced care decisions/management. All abnormalities are validated and certified by best practice multiplanar and dimensional imaging and multimodality investigation (multiple measurements/calculations). Imaging is enhanced by best practice modeling of heart function and disease with dimensional analytics. Research is dramatically improved by making historical ECHOs/imaging/clinical profiles always available for newer/enhanced and prospective analytics. Correct and enhanced imaging, imaging and data analytics are automatically triggered and do not depend on sonographer/echocardiographer/system expertise. Accordingly, the labs are aware of best practices. Research is best practice and better incorporated in clinical imaging/practice. Certified and validated structural/functional/hemodynamic profiles and disease analytics should finally lead to standardized inter-modality analytics and guaranteed comparability and dimensional analytics for all imaging, including, but not limited to, ECHO, CT, MR, SPECT, PET, and electrocardiography.

An outline of the smart ECHO system 100 includes: i) Sonographer feedback, ii) Add on edge computing hardware and software system/interactive network for Intelligent acquisition of images and triggers/initiation of specialized testing, iii) Interactive real-time display of models, results and data analytics by add-on systems, iv) AR headsets/best practice display for interactive display, dimensionally correct image display, conflict/pathology display, dimensional display, modeling/hemodynamic profiling and image display, v) Smart reporting-confirmation of structural/functional/hemodynamic profile, staging of disease/disease analytics, change mapping and disease analytics, vi) IT for immediate uploading of old studies, vii) Complexity analysis for reporting, viii) Communication to providers, ix) Intelligent modeling, x) Matrix of interdependence, xi) Conservation of mass, xii) Best practice development and incorporation, xiii) Machine learning/deep learning of methods including constant refinement, xiv) Regeneration of normal and standards and measures of severity, xv) Protocol standardization/refinement/new methods, xvi) Research innovation, xvii) Intelligent associations based on significant findings, xviii) Refinement of dimensional analysis (rhythm, status, condition), xix) Intelligent processing of legacy studies, xx) Artificial intelligence, xxi) Artificial intelligence to change concept of computing, and xxii) New opposite paradigm: Computer to learn need of provider and how to help provider, this includes unlimited access to infinite programing/programs/apps/AI/ML/DL/DA/PA.

The Sonographer feedback includes: a) Sonographer, b) Heart/disease Model, c) Front end processing of appropriate use criteria (AUC) processing of indication at the time of ordering to guarantee test appropriateness and prevent redundant unnecessary tests, d) Data sets, e) Observations, f) Grades of severity, g) Extent, h) Complexity, i) Machine supported image acquisition, and j) Edge computing add on system to ECHO machine for interactive imaging.

The add on edge computing hardware and software system/interactive network for Intelligent acquisition of images and triggers/initiation of specialized testing includes: a) Partnership between system and sonographer, b) Anatomic intelligence targeted Doppler, c) Sonographer initiated and Machine optimized Doppler imaging, d) Continuous conservation of mass feedback, e) Pathology triggered specialized testing, f) Best practices incorporated for machine set up/methods/techniques, g) Best practices specific for all possible pathology, h) Confirmation of pathology by multiple methods, i) Global implications of findings immediately displayed, j) Sonographer always aware of matrix of interdependence, k) Data driven reporting as sonography/machine perform study, and l) Sonographer/Machine learning for best and most efficient acquisition of images.

The Interactive real-time display of models, results and data analytics by add-on systems includes a) Start with current state of the art system computer/hardware, b) All m-mode/2D/3D/4D/dimensional/strain/color & spectral Doppler/speckle tracking methods imbedded with no limits to future innovation, c) No display constraints. Technological limitations of screen resolved, d) Infinite flexibility of display and true display of image (single dimensions/2D/3D/4D/additional temporal/conditional dimensions), e) M-mode, 2D/3D, phased array/volume transducers/vascular annular array and TEE probes. Smart XYZ plane imaging, 3D anatomic driven 2D and m-mode/Doppler/speckle imaging, f) Real-time image and data analytics/feedback to sonographer/protocol alterations, g) Intrinsic awareness of vital signs, O2 saturation, electrolytes, Hgb, TSH, WBC, rhythm, sed rate, unlimited lab resulted and serial changes, heart rate/RR interval, ORS duration, QRS complex, ST segment deviation, T wave deviations, PR interval/QT interval, relevant micro-changes in electrocardiogram, clinical symptoms, exam findings, other and whole body imaging, demographics, access limitations, and socio-economic issues, h) Built in data analytics and supervised machine learning for tailored imbedding of foundation of best practice methods based on learning from database of studies acquired and measured in according to best practices and complete structural functional and hemodynamic profiles and datasets, i) System/supervised machine learning (unique set of 20000-30000 best practice correctly annotated and analyzed ECHOs stress, TEE and interventional ECHOs for last 4 years)/deep learning to continuously improve methods and data analytics/processing, j) AR/holographic for sonographers/echocardiographers doing the rest ECHOs, stress, interventional, procedural and optimization ECHOs/TEE ECHOs, k) Sonographer always seamlessly connected to echocardiographer (audio/video/display/map/face to face), l) Predictive analytics of typical associated/incidental findings associated with moderate/severe findings, and m) Connection to HD TV in patient room to show study to patient or providers.

The augmented reality (AR) headsets/best practice display for interactive display, dimensionally correct image display, conflict/pathology display, dimensional display, modeling/hemodynamic profiling and image display includes a) Holographic display vs augmented reality, b) Limitless display of model information, hemodynamic profile and any dimensional analysis at same time as active image, c) Current image always in center of visual field with image associated models of structure, function and hemodynamics, d) Display alerts of relevant pathology and changing dimensional analysis, e) Images always displayed in dimensionally correct best practice format, f) Measurements and associated analytics displayed with images, and g) Interactive verbal and visual communication between sonographer and computer/network and system.

The Smart reporting—confirmation of structural/functional/hemodynamic profile, staging of disease/disease analytics, change mapping and disease analytics includes a) Data driven reporting, such as correct grading of severity, extent, and complexity, b) Final conflict resolution/data validation based on physics/matrix mathematics/confidence of measurements, c) Final confirmation of correct and complete use of specialized testing, d) Mathematics/physics based validation of volume data/matrices of data inter-relationships, e) Appropriate initiation of dimensional analysis, such as i) Atrial arrhythmias, ii) Ventricular arrhythmias, iii) Stress staging, iv) Interventional ECHOs, v) Interpretation at same RR interval for afib/aflutter cases, and vi) BP specific reporting in monitored patients, f) Final anatomic description, g) Final reporting of complete extent and complexity of disease, h) Tailoring report to sophistication/training of provider, i) Prioritized communication, j) Confirmation of temporal differential mapping/presentation of changes/data extraction from prior studies, k) Machine learning/refining of acquisition/processing protocols, data analytics and databasing. Appropriate updating of standards and best practices, l) Development and incorporation of new methods and processing, m) Tailored mode of communication with provider, and n) Confirmation of appropriate use criteria (AUC) processing of indication to guarantee test appropriateness and prevent redundant unnecessary tests.

The smart ECHO system 100 is configured to access uploaded historical studies (e.g., historical ECHO results) including a) A complete database of all digital/DICOM formatted studies, b) State of the art high speed network/edge computing with direct connection of sonographer/ECHO with server, c) Data and related/appropriate image extraction from AI processed ECHOs/hemodynamic profiles. Side by side display of legacy and dimensional smart images with imbedded measurements/associated analytics, display of changes, and image display based on structure and function, d) Immediate display and high fidelity comparation of the new and old image with complete high-fidelity comparative analysis, e) Simultaneous storage and uploading of images to machine and server, f) Intelligent storage of data and complete processing to state of the art/best practices/best data analytics, g) AI/machine learning/deep learning to ensure best practice acquisition and processing, h) Wireless network, i) Hard wired, j) Ordering triggered immediate uploading of studies to current/active server, k) Hospital system and network uploading of prior studies or CD/media based up loading prior studies for anatomic assessment/hemodynamic profiling and disease modeling/severity/extent/complexity, l) HIPAA compliance, m) Immediate identification and awareness of differences in clinical condition, n) Intelligent learning of location of possible comparable studies, and o) Immediate cross referencing with surgical and interventional/clinical reports for automatic incorporation in reports and awareness by sonographers/echocardiographers/intermediate staff.

The Complexity analysis for reporting includes a) Investigation for all typical findings associated with moderate to severe findings, b) Severity standards drive reporting, c) Continuous AI/machine learning and deep learning of typical disease profiles and possible findings exacerbating patient condition, d) Change differential mapping of disease/condition/hemodynamic profiles/anatomic information, e) Relevant reporting and awareness to imagers of disease acceleration rather than linear velocity change, f) High risk conditions identified and reported to sonographer and echocardiographer/intermediate staff, g) Immediate order sets uploaded when additional noninvasive or invasive imaging needed, h) Immediate communication of high risk to ordering provider and uploading of order sets for surgical consultation(s), i) Communication to providers, j) Tailored to provider, k) Communication preferences imbedded, l) Guaranteed answering of all clinical questions, m) Management recommendations if preferred, and n) Consultation recommendations if preferred.

The Intelligent modeling includes a) Moderate and severe abnormalities as triggers, b) Appropriate specialized testing, c) Awareness of common associations, d) All pre-existing findings verified, e) Common associated anatomic and physiologic/pathophysiologic abnormalities and grades of findings, f) Investigation for possible findings based on clinical changes, stress and interventions, g) Moderate to severe anatomic abnormalities to trigger investigation of Doppler/hemodynamic disturbances, h) Doppler abnormalities and type to trigger investigation for anatomic abnormalities, i) Heart rhythm abnormalities to trigger investigation for cases, j) Interventions to trigger investigation for possible volumetric and functional changes (wall motion, strain, stress, valve function, systolic and diastolic function, resistance, compliance and pressures/gradients, k) Interventional methodologies to trigger investigation for expected findings/investigations (trans-septal to trigger screen for shunts and ASDs, Mitra clips trigger compete volumetric and functional assessment/valve stenosis screening, plugging PVL/VSD/fistulas/TAVR/TMVR appropriate screen, and l) Sx with nondiagnostic/negative studies to trigger dimensional analysis/stress/provocative testing.

The matrix of interdependence includes a) All valves, and chambers linked by volumes and flows. M-mode, 2D, 3D, 4D, spectral and color Doppler, b) Anatomy linked by volumes and flows, c) Resistance only accurate when volume and flow correct, d) Anatomy validates Doppler/physiology, e) Doppler/physiology validates anatomy, f) LV and RV stroke volumes the same except with beat to beat variation in constriction/overall minute volumes the same, g) Dynamic flow associated with mean areas and laminar flow with max single valve areas, h) Pressures, flows, resistance volumes always linked. i) Stress directly related to pressure and size and inversely related to thickness, j) Velocity, pressure linearly related to flow at low resistance and geometrically related at high resistance, k) Time integrals of velocity reflect flow, l) Gradients related to flow, m) Net volume of flow in and out of heart reflected Doppler and anatomy of all valves and chambers (LV and RV stroke volumes Doppler validated by net flow through heart and regurgitant volumes of associated valves), n) Pressure development related to stiffness, relaxation, scarring, dyssynchrony, timing of events, restrictions, o) Valve function related to impendence, pressure, vascular/chamber stiffness/resistance, intrinsic properties and anatomy of the valve, and p) Chamber contraction related to pressure, wall stress, strain, muscle mass, calcium, hormones, support structure/infrastructure. AI to be able to develop and track pressure volume loops in real time.

The conservation of mass includes a) Flow entering the heart equals the flow exiting the heart, b) Continuity equation, c) The concept connecting anatomy and physiology, d) Error and conflicts due to measurements at different sites, e) Resolution by special memory and awareness, f) Valve size validated by volume/flow data (annulus and Doppler at annulus, LV and RV stroke volumes Doppler validated by net flow through heart, chambers associated shunts, and regurgitant volumes of associated valves). Cross validation timing, max and mean areas by m-mode, 2D, 3D, 4D, spectral Doppler, g) Shunts, h) Valve stenosis, i) Aneurysms, j) Valve regurgitation, k) Fistulas, l) Relevant factors for validation of valve size, m) Relevant factors for validation of diastolic function, n) Relevant factors for validation of valve resistance and chamber impedance, o) Relevant factors for validation of pulmonary and systemic vascular resistance, p) Relevant factors for validation of chamber volumes and systolic function, q) New analytics of volume validated m-mode, 2D/3D/4D motion and spectral and color Doppler strain, strain rate, displacement, patterns of flow, timing of flow, rates of change, acceleration, deceleration, turbulence, intensity and flow patterns extract unrecognized relevant data, and r) Improved Doppler and anatomic information by awareness/correction for heart/structure motion/displacement in space.

The Best practice development and incorporation includes a) Machine settings always optimized, b) Protocols always optimized, c) Auto-feedback to practices of sonographer, d) Best practice use of specialized testing, e) Best practice use of contrast for 2D/3D/4D imaging, f) Best practice use of strain imaging, g) Best practice se of XYZ plane imaging, h) Best practice use of multiplane imaging, i) Best practice use of multiplane color Doppler imaging, j) Best practice use of 3D/4D/2D/m-mode imaging, k) Best practice use of spectral Doppler imaging, l) Best practice use of spatial memory for combining anatomic and Doppler imaging, m) Best practice use of dimensional imaging, n) Optimized gain, compression, depth, magnification, sweep, scale, baseline, gate size, focus and filter settings for each Doppler image and plane, o) Optimized use of contrast for Doppler imaging of valve stenosis and regurgitation (quantitation), p) Best practice use of extended imaging (aorta) based on clinical history. q) Best practice video-densitometry assessment of myocardial perfusion. ML of best protocol based on comparative analytics with angiographic FFR and IFR data, r) Best practice use of anatomic and Doppler/physiologic/pathophysiologic imaging for commonly associated findings (disease extent and complexity) with any moderate to severe findings, and s) Best practice comparative imaging and analysis for legacy/serial and dimensional studies for correct predictive analytics, complementary data analytics/enhanced information, risk assessment, guarantee appropriateness, enhanced modeling, improved disease detection/assessment/prognostication, change/differential/acceleration mapping, new risk assessment and improved disease staging/modeling/rate of change.

The Machine learning/deep learning of methods utilize constant refinement including a) Initial learning of best practices/data analytics from the best sonographers (machine settings and methods), modeling based structural echocardiographers and ASE/physics-based imaging standards maximizing quality, reliability and accuracy, b) Best practice for feedback to sonographers and display of models of heart function, c) Best practice for machine assisted sonographer intelligent acquisition of anatomic and Doppler images, d) Spatial memory for any location specific acquisition and labelling of complementary data for appropriate calculations/secondary/tertiary/quaternary data analytics, e) Initial learning of best practice methods and editing/anatomic and Doppler data analytics of advanced structural echocardiographers trained in volumetric imaging, conservation of mass/physics and mathematics in imaging, internal validation/certification of data and modeling of disease extent/severity/complexity, f) Automated maximum, mean, modal, minimum, color enhanced, contrast enhanced measuring of data. New real-time pressure/strain/strain rate/stiffness loops of function and correct measurements of rates of change, acceleration, change and slope imaging, g) Predictive analytics for typical associated of findings with moderate to severe abnormalities, h) Machine learning/deep learning with each additional processed study, i) Constant updating of best measurement methods, anatomic associations, internal checks, volume standards, validation methods, data analytics, machine settings, acquisition sequence, building/scope of the heart function/disease models, communication of results/implications to sonographer and method (verbal/text/image)/style/volume/timing/wording of the interaction between machine and sonographer, and j) Updating of the feedback or automation of correct anatomic plane, sample volume placement, cursor placement, reporting, acquisition settings, and data analytics.

The regeneration of normal and standards and measures of severity includes a) Initial standards based on current standards, b) Intelligent information extracted from clinical notes and other assessments/imaging/angiography/catheterizations/hemodynamic data, c) Confirmation or correction of clinical information and resolution of conflicts, d) Every patient/ECHO to be added to databases of normal values and abnormal/disease, e) Any demographic for normal values and disease grades, f) Hospital system and network wide databases where the ultimate goal is an international database, g) Findings missed on prior images/ECHOs and new associations, h) Collaborative databasing and quality updating with smaller hospitals and offices.

Improvements to protocol standardization/refinement/new methods provided by the smart ECHO system 100 include: a) Point of care limited ECHO screen for low risk patient populations/clinical predictive analytics, b) Complete protocol when appropriate for certified/validated complete structural, functional and hemodynamic profile, c) Ongoing refinement and distribution of the partnership between ECHO machine and sonographer/echocardiographer/data and image analytics, d) Best practices easily added and incorporated, e) Retrospective analysis of legacy studies and prospective analysis of new studies, f) Rapid validation of new measurements/methods and analytics/calculations/derivatives/mathematical transformations, g) Testing of analytics on legacy and prospective studies, h) Constant refinement of intelligent image acquisition and acquisition protocol, i) Protocols always updated to best practice standardized protocol, and j) All reports tailored to complete hemodynamic/structural/functional profiles, certified/validated/reproducible data, standardized criteria and observations, best practice comparative and dimensional analytics and data driven reporting.

Improvements to researching provided by the smart ECHO system 100 include: a) Refinement of acquisition protocol, b) New imaging methods including New frame by frame analysis of 2D/3D/4D images and comparative analytics, as well as Pressure-volume and strain/strain rate/wall stress/stiffness/impedance/resistance-volume loop measurements, c) New dimensional analytics based on continuous BP devices, heart rates, heart rhythms, exam findings, clinical findings, electrolytes, metabolic, inflammatory, blood. Liver, other laboratory abnormalities, radiologic and imaging findings, d) Disease/dysfunction burden bases on rate, rhythm, QRS complex, conduction abnormality, clinical parameter, coexisting disease, potential intervention, prediction of effects of interventions, e) New high frequency and pattern analytics of m-mode datasets, f) Compensation for motion of heart structures in space, g) New dynamic heart spatial motion compensated Doppler imaging, such as Precise placement of pulsed Doppler sampling volumes at same site as anatomic measurements with maximum/modal/minimum pulsed Doppler velocity and velocity time measurements, h) Dynamic multiplane imaging, i) Mean valve areas as new marker of valve function especially AV valves, j) Validation of new methods based on retrospective analysis of legacy studies and prospective analysis of new studies. k) New much larger hospital, system-wide, state-wide, nation-wide, international databases of anatomic and hemodynamic profiles for improved prediction of prognosis/adverse outcome and parameters for screening for/following conditions/diseases/disorders, l) Research on scale never seen before or previously considered (thousands to millions), and m) Easy reprocessing of studies to near immediate validation or invalidation of new hypotheses.

The intelligent associations based on significant findings includes a) Coexisting findings with congenital heart disease/common interventions/repairs, b) Valve disorders (anatomic and disease variants) and structural conditions, c) Clinical conditions/findings/disorders and expected findings based on severity and complexity, d) Clinical decompensation and expectations, e) Rhythm abnormalities and structural/functional findings/abnormalities, f) ECG abnormalities and structural/functional findings/abnormalities, g) CXR/radiologic/other modality findings and structural/functional findings/abnormalities, h) Reliable exam and structural/functional findings/abnormalities, i) Intelligent processing of clinical notes and other tests/imaging/findings, and j) All imaging to be complementary.

The refinement of dimensional analysis such as rhythm, status, and condition, includes a) Measurements embedded with clinical/temporal/dimensional/conditional/interventional data, b) Complete datasets for each clinical/temporal/dimensional/conditional/interventional condition for secure validation, c) Current model, d) Change presentation. Alterations in spatial cardiac motion and excursion, e) Temporal differential mapping of disease, f) Temporal disease acceleration, g) Intervention change imaging and extent, h) Intervention differential mapping of changes, i) New exacerbating condition and change/differential/acceleration mapping, and j) Immediate awareness of relevant changes/high risk parameters relative to prior study/imaging and interventions.

The intelligent processing of legacy studies includes a) All prior digital/DICOM studies eligible, b) Smart ECHO processing to further expand database and dimensions of disease, c) Triggered SMART analysis or even prospective reanalysis of all prior amenable studies, d) Prospective reanalysis of all prior amenable studies for automatic dimensional analysis/new standard of disease complexity, e) Studies not previously smart imaged and processed to be intelligently processed/analyzed by even more sophisticated data analytics with enhanced anatomic and Doppler pattern and location/method specific recognition, f) Complete prior anatomic and hemodynamic profile available to sonographer before starting exam, g) Sonographer/echocardiographer always aware of any significant pre-existing conditions, h) Real time sonographer awareness of changes in condition or changes associated with interventions or clinical/dimensional changes, i) Change mapping for immediate recognition, j) Differential mapping for immediate recognition, and k) Acceleration mapping for immediate recognition of high risk.

There are two major components of embodiments disclosed herein include: a) AI based smart ECHO lab as described herein, and b) Smart ordering and immediate appropriate use criteria (AUC) processing.

The AI based smart ECHO lab includes 1) Add on interactive analytic edge computing system, 2) New best practice display, analytic, interactive stations, 3) two-way network, 4) AR based intelligent acquisition and front-end data analytics/continuous as needed communication between sonographer and echocardiographer, 5) real-time modeling and report generation, 6) real time identification of pathology/normal, 7) unlimited dimensional analysis, 8) early identification of high risk, 9) intelligent legacy analysis, 10) intelligent and complete structural and hemodynamic modeling/profiling, 11) front end intelligent comparative analysis, 12) dimensional/differential/velocity/acceleration mapping of disease and changes and near immediate communication of final report to providers, 13) dimensional/differential/velocity/acceleration mapping of disease and changes and near immediate communication of final report to appropriate consultants, 14) Automated real-time search of medical literature and visual (pictorial and video) for anatomic/structural abnormalities/anomalies, 15) Intelligent comparative analysis with personal/hospital/system/national/society databases and literature for improved diagnostic accuracy, 16) AR link to PCP when seeing patients for input from and empowerment of PCP in care of patients (involving the provider with the greatest legacy knowledge of patient, real-time awareness of care and design of follow up care), 17) Imbedded preferred methods of communication (AR visual/verbal/images and mixed) emergency links when appropriate, and 18) Simultaneous maximum workflow and quality.

The Smart ordering and immediate appropriate use criteria (AUC) processing includes 1) Best practice ordering, 2) Data analytics, 3) Predictive analytics, 4) No redundant studies, 5) Easy and intelligent cross comparative/dimensional analysis, 6) Elimination of studies with no downstream utility or consequence, 7) All studies at best practice level, 8) Minimization of errors, 9) Better outcomes, 10) Improved cost-effective care, 11) Highest quality testing, 12) Maximal speed and accuracy of all tests/care, 13) All studies immediately tended for disease dimension, velocity and acceleration mapping, 14) Reduced cost, 15) Increased speed and continuously optimized care, 16) Constant communication with intelligent EMR to determine need for testing, if studies impact care, ensure cost effectiveness and truly define best practice ordering/interpretation/appropriate use. AI to truly define AUC base on clinical impact/effects, 17) All related tests (results/images) automatically imbedded, 18) Results seamlessly sent to ordering provider/hospital caregiver and PCP, 19) Dramatic reduction or elimination of malpractice, 20) Two-way LAN and IT the key to AI in health care. Machine learning/deep learning/data analytics and predictive analytics at all levels of care/assessment and follow up, 21) Imbedding of all studies in orders, 22) No computer screens. VR/AR for final analysis and reporting. AR for all staff/easy of processing/ordering/test, 23) performance/completion/communication and medical/interventional care, 24) The fastest and highest quality with state-of-the-art IT/LAN/two-way network/intelligence at every step, and 25) AI/machine learning/deep learning and literature/guideline best practices continuously refined and updated.

The long term benefits provided by the smart ECHO system 100 include: a) New dimensional awareness of disease extent/severity/complexity and prognosis/best practice care for any demographic, b) Normalcy defined for any demographic, c) Every study and care everywhere at best practice level, d) Malpractice minimized, e) Healthcare costs reduced by 10-25%, f) Research on scale never seen before diagnostic prognostic power for better/more cost-effective care/improved outcomes/targeted and better tailored care for all, g) Research based on massive databases, h) No need for core labs, i) Better monitoring of patients in rehab facilities/long care facilities/SNF/assisted living and independent living for care issues/abuse and management/end of life care, j) More rapid development and refinement/optimization of new technologies/methods/imaging/therapeutics. k) AI for best practice CME for physicians such as Links and trigger for CME imbedded in system may obviate need for travel and hotels which may provide More effective and efficient use of travel by physicians and staff, and l) Greater depth and access for staff in all services and labs through virtual learning.

Echocardiography is a powerful tool for screening and monitoring for heart disease. ECHO can differentiate risk, facilitate care, reduce cost, and improve outcomes but often utilizes significant time and personnel. ECHO now rarely meets best practice standards due to time and workflow demands. Reimbursement incentivizes speed over quality time-intensive validated analytical reading resulting in incomplete ECHOs with errors, conflicts, and missing data, and poor care and outcome. Healthcare demands better.

Artificial intelligence, machine learning, and deep learning promise to improve precision, accuracy, comparability, analytics, work environment and workflow but application have been limited. ECHO is the most demanding of all imaging with up to four-dimensional imaging of motion, structure, thickness, function, excursion, radial strain, pixel tracking for strain and strain rate, anatomy, relationships of structures, size, dimensions, areas, volumes, and mass. Tissue Doppler measures displacement, strain, strain rate, velocity, acceleration of structures, and muscle walls. Blood pool Doppler measures in up to 4 dimensions flow/volume and timing of flow, velocity, direction, flow complexity, acceleration, flow patterns and penetration in chambers, valve/orifice dimensions/flow patterns, and shunts. Mathematical calculations/transformation convert Doppler blood flow into area, pressure gradients, flow rates and volumes. Simultaneous structural data and integrals of time flow velocity calculate stroke volumes, leak volumes, obstruction, resistance, and impedance. Various contrast agents enhance structural and Doppler imaging for blood flow in chambers and myocardium.

The application of AI/ML/DL to ECHO has been limited with substandard accuracy and significant potential for error but now is no longer optional. AI/ML/DL of images and intelligent acquisition and data processing are the only solution to quality demands and workflow/logistics. Errors due to non-validation are the limitation to AI/ML/DL in ECHO. Precise validation and resolution of obstacles may be the gateway to all other imaging for best quality and workflow.

AI, ML and DL based on erroneous, nonreproducible, non-validated training principles not only causes but magnifies errors of interpretation, disease grading and analytics. Current accuracy is not sufficient for automation. This project is essential to the application of AI/ML/DL to ECHO and imaging. Smart ECHO validation is performed based on a series of more than 5,000 ECHOs manually interpreted by an expert echocardiographer with imbedded best practice image recognition, measurements; data, disease, dimensional and comparative analytics. All manually validated ECHOs are cross validated by the laws of conservation of mass, mechanics, flow dynamics, and pressure volume flow and new structure volume flow relationships. The smart ECHO system 100 may build in the best practice image recognition and structural functional analytics in the setting of robust matrices of interdependence and cross validation principles. Manual data/results may be HIPPA mined into large database along with re-interpretation results by AI/ML/DL image, disease, dimensional and comparative analytics. Studies may be processed in groups of 100 with complex database analytics to identify and correct image recognition, analytic and grading errors, and conflicts. The process may improve validation and accuracy of all analytics.

The smart ECHO system 100 meets the needs for both maximum quality and logistics: a) smart ECHO intelligent interactive image acquisition, b) Machine/network development for state-of-the-art augmented reality display, and c) Enhance/refine communication/workflow analytics for highest impact, quality, and workflow.

The smart ECHO system 100 has several advantages including that it improves quality of care; changes research; reduces cost including unnecessary admissions and lengths of stay; improves intervention timing/impact; improves productivity, cost- and time-efficiency; reduces malpractice and errors; improves access best practice imaging; reduces burnout, improves staff quality of life and personal time; and directs prevention.

Echocardiography (ECHO) is a powerful tool for screening and monitoring for heart disease. ECHO is capable of up to 4-dimensional images of the heart for structure, mechanics, flow dynamics, myocardial blood flow, and hemodynamics; ideal for disease profiling and modeling, and complex disease analytics to differentiate relative roles and interdependence of diseases. Results are reproducible and comparable, contain cost, improve outcomes, differentiate low from high risk, and facilitate/refine care, prognosis, early detection, and cardiac interventions. Downstream effect is improved disease management, outcomes, and utilization of interventions (1-24).

ECHOs able to differentiate risk, facilitate care, reduce cost, and improve outcomes often includes significant time and personnel. ECHO rarely meets best practice standards due to time and workflow demands. Reimbursement incentivizes speed over quality time-intensive validated analytical reading resulting in incomplete ECHOs with errors, conflicts, and missing data, and poor care and outcome. Quick qualitative assessment is the standard due to time and workflow/volume demands. Volume/RVU reimbursement incentivizes speed reading/multitasking/cursory reading. Speed reading produces greater reimbursement. Sonographers are doing more ECHOs in shorter visits. High sonographer and professional staff stress cause high turnover, poor images, and nonsensical measurements/calculations. Sonographers are not aware of normal values, grades, data interdependence or expected coexisting findings. ECHOs often conflict with gold standards and invalidate principles of conservation of mass, mechanics, flow dynamics, pressure volume flow and new structure volume flow relationships. Standards for normal/abnormal results are unknown with no consideration for demographics. In conventional practice, readers and sonographers frequently dismiss or abbreviate interpretive tasks in order to justify accelerated review (“speed reads”). Such practices, while aimed at efficiency, can compromise diagnostic quality and reproducibility. The healthcare industry, however, demands improved systems and methods that provide both accuracy and timeliness, thereby overcoming the deficiencies of speed-focused interpretation.

Artificial intelligence (AI), machine learning (ML), and deep learning (DL) promise to improve precision, accuracy, comparability, analytics, work environment and workflow but application have been limited and complicated. ECHO is the most demanding of all imaging with up to four-dimensional imaging of motion, structure, thickness, function, excursion, radial strain, pixel tracking for strain and strain rate, anatomy, relationships of structures, size, dimensions, areas, volumes, and mass. Tissue Doppler measures displacement, strain, strain rate, velocity, acceleration of structures, and muscle walls. Blood pool Doppler measures in up to four dimensions flow/volume and timing of flow, velocity, direction, flow complexity, acceleration, flow patterns and penetration in chambers, valve/orifice dimensions/flow patterns, and shunts. Mathematical calculations/transformation convert Doppler blood flow into area, pressure gradients, flow rates and volumes. Simultaneous structural data and integrals of time flow velocity calculate stroke volumes, leak volumes, obstruction, resistance, and impedance. Various contrast agents enhance structural and Doppler imaging for blood flow in chambers and myocardium. Intelligent labelling of images may better display interrelations and complexity.

The application of AI/ML/DL to ECHO has been limited by substandard accuracy and significant potential for error but now is no longer optional. AI/ML/DL of images and intelligent acquisition and data processing are the only solution to quality demands, disease analytics, care recommendations and workflow/logistics. Errors due to non-validation are the limitation to AI/ML/DL in ECHO. Precise validation and resolution of obstacles may provide a gateway to all other imaging for best quality and workflow.

AI, ML and DL based on erroneous, nonreproducible, non-validated analytics not only causes but magnifies errors of interpretation, disease grading and analytics. Current accuracy is not sufficient for automation. The smart ECHO system 100 validation is, in some embodiments, based on more than 5,000, more than 6,000, and/or more than 10,000 ECHOs manually interpreted by an expert echocardiographer with imbedded best practice annotation and measurements; data, disease, dimensional, and comparative analytics. All manually interpreted ECHOs are validated by the laws of conservation of mass, mechanics, flow dynamics, pressure volume flow, and new structure volume flow relationships. The smart ECHO system 100 builds on the best practice image recognition and structural functional analytics in the setting of robust matrices of interdependence and cross validation principles. Manual data/results are HIPPA mined into large database along with re-interpretation results by AI/ML/DL image, disease, dimensional and comparative analytics. Studies are processed in groups of 100 with complex database analytics to identify and correct algorithm based image recognition, image analytics, grading errors, and conflicts. The process guarantees validation and accuracy of all analytics.

The smart ECHO system 100 meets the needs for both maximum quality and logistics: a) Smart ECHO intelligent interactive image acquisition, b) Machine/network development for state-of-the-art augmented reality display, and c) Enhance/refine communication/workflow analytics for highest impact, quality, and workflow.

The smart ECHO system 100 has several advantages including improved quality of care; timely/real time best practice updates; more innovative research; reduced cost including unnecessary admissions and lengths of stay; improved intervention timing/impact; improved productivity, cost- and time-efficiency; reduced malpractice and errors; improved access to best practice imaging; reduced burnout, improved staff quality of life and personal time; and directed prevention. Net effects may also include increased margins and less need for coding, monitoring and risk management.

Echocardiography with artificial intelligence (AI) succeeds with simultaneous maximum quality and workflow/logistics. Patients are guaranteed reproducibility, accuracy, confidence, completeness, and inter-modality (all ECHO/MR/CT/nuclear) comparability. Reports can be finalized and communicated as the imaging is done and may provide an intelligent servo-mechanism for AI/ML and DL to help the sonographer according to the experience and knowledge to make quality and workflow/revenues symbiotic.

Sonographers cannot by themselves meet the current imaging, processing, and interpretive demands of ECHO. Workflow and quality demands can only be met with current and worsening time constraints by a team/partnership approach. The current highest quality standard is the Mayo clinical team approach can help (10 reading groups per day for 200 ECHOs with teams of one to two echocardiographers for final quality assurance/final reporting before patient leaves the ECHO lab, two super-sonographers skilled in measurements and data processing/image and data quality/analytics, and three sonographers trained in protocol-based image acquisition with weekly personal feedback on quality) but is not financially feasible in other labs. The smart ECHO system 100 with validated AI/ML/DL and analytics improves on the above system.

Multiple short and long term benefits provided by the smart ECHO system 100 include, but are not limited to a) New dimensional awareness of disease extent/severity/complexity and prognosis/best practice care for any demographic, b) Normalcy defined for any demographic, c) Every study and care everywhere at best practice level, d) Highest quality care at maximum workflow, e) Outcomes research for better/more cost-effective care/improved outcomes/targeted and better tailored care for all, f) Research based on massive databases/No need for core labs, g) New awareness and disease classification, h) Minimization of physician staff burnout/More time for direct patient contact, i) Higher volumes and shorter length of stays. Minimizing unnecessary admissions/testing, j) More efficient, cost effective, timely and higher quality patient care, k) Cost containment, greater margins, improve staff and professional staff productivity, highest quality as highest producers, l) Reduced physician and staff burn out, minimal homework, greater time directly for patient care, reduced computer time, better home and family life, greater respect from administration, healthier lifestyle, more time for community/school relationships, more time for research without compromising clinical productivity, m) Enhanced ability of best providers to care for patients and have a family life, n) Higher margins for higher staff/faculty pay, better retention, and more money for R and D, o) Enhanced local and national reputation, research/academic and clinical productivity, and p) Enhanced status in AI/national leadership role in science/business/healthcare.

The smart ECHO system 100 achieves the needed quality standards for AI in ECHO an imaging by the rigorous validation process proposed against the gold standard of the unique profile of each ECHO/its unique single solution to matrix of mathematics and the database/archive of ECHOs manually measured and interpreted with all the validation methods. This archive is a true gold standard since all of the studies have been validated with clinical, angiography, invasive hemodynamic and other imaging studies. At least some of the technical benefits of the embodiments described herein are based on developing and refining the analytics based on the entire ECHO study. The smart ECHO system 100 builds in potential/expected findings to be triggered by ordered indication (intelligent probability), clinical diseases, patient status (stable/critical/shock/arrest/severe HTN, etc.), and expected associated findings based on any moderate or severe finding. Thereby, prior knowledge/models of disease can be used to enhance the probability of detecting the true extent, scope, and complexity of the abnormalities and disease. A feedback loop process of individual study and group database/analytic assessment of visual analytics, image extraction analytics, ML and DL analytics, image and Doppler measurement analytics, structural recognition analytics, disease management analytics, data driven reporting analytics, comparative analytics, and dimensional analytics in groups of 100 studies.

The smart ECHO system 100 may then a) Validate and perform legacy analytics of ECHOs read by current practice methods with data analytics to identify conflicts; unrecognized measurement, processing, grading, reporting errors; common errors; beneficial effects of legacy study analytics; potential clinical issues relating to legacy analytics; disease and overall magnitude/value of corrections and enhanced analytics, b) Imbed the best visual analytics, image extraction analytics, ML and DL analytics, image and Doppler measurement analytics, structural recognition analytics, disease management analytics, data driven reporting analytics, comparative analytics and dimensional analytics in the ECHO machine, c) Develop/refine the smart ECHO machine with AR display to establish the intelligent acquisition partnership of the machine/AI/real-time analytics with sonographers (TTE and stress studies) and echocardiographers/trainees (fellows)/trained professionals (TEE and interventional/point of care studies), d) Develop/refine the smart ECHO multidimensional/multidirectional intelligent network for real-time comparative analytics as imaging is done to link the sonographer, staff fellows, professional staff and echocardiographer. Develop the options of real-time reporting, real-time identification/communication of high-risk conditions, and a best practice environment in which sonographers and fellows/trained professionals are always supported and linked to echocardiographers or director of the lab, e) Develop/refine smart ECHO to trigger dimensional analytics based on heart rate, rhythm, changes in patient status condition, interventions, and exercise/pharmacologic stress, f) Develop big data databases for enhanced computer science/AI/BL/DL methods and benefits/issues, enhanced clinical research and expanded outcomes research/better disease monitors and analytics g) Develop/refine the smart ECHO multidimensional/multidirectional intelligent network with HIS best practice reporting and relevant image links for providers, h) Develop/refine the smart ECHO multidimensional/multidirectional intelligent network with HIS/EHR for comparative analytics with other imaging, and i) Develop/refine the smart ECHO multidimensional/multidirectional intelligent network with HIS/EHR for AUC (appropriate use criteria) based ordering of ECHOs, reduced ordering errors, and automated imbedding of other imaging reports and relevant pictures/movies.

The work context of the smart ECHO system includes modeling, validation, cross validation, interdependence, conflict resolution, intelligent acquisition, feedback, and support.

AI is dependent on validated image recognition, data extraction, measurements, and analytics. AI without validation exacerbates erroneous reporting. Accordingly, the smart ECHO system 100 provides the needed quality standards for AI in ECHO an imaging by the rigorous validation process proposed against the gold standard of the database/archive of ECHOs manually measured and interpreted with all the validation methods. This archive is a true gold standard since all of the studies have been validated with clinical, angiography, invasive hemodynamic, and other imaging studies. A feedback loop process may be used including an individual study and group database/analytic assessment of visual analytics, image extraction analytics, ML and DL analytics, image and Doppler measurement analytics, structural recognition analytics, disease management analytics, data driven reporting analytics, comparative analytics, and dimensional analytics in groups of 100 studies.

The processing of ECHOs is streamlined by maximum confidence in the primary, derived data and analytics. There is more time to focus on quality/direct patient interaction, rare corrections, no typing, better disease analytics, data driven reporting, better impact on providers by maximum clarity and tailored conclusions, complete data sets and enhanced clinical reporting quality. Big data sets and enhanced analytics may generate/reassess normalcy and grades of disease extent complexity and severity for all demographics. Intelligent front-end image acquisition and processing may guarantee best practice imaging methods are imbedded and updated constantly. The process may supplant any need for core labs for future research by best practice imaging and analytics for all patients. The entire network of ECHO machines (nodes) can be used to test and validate new technology/analytics irrespective of location or sonographer/echocardiographer knowledge and experience. Sonographer retention may improve leading to enhanced/maximal sonographer quality. Standardized, accurate, and complete reports and analytics may guarantee lab accreditation.

Resolution of HIPAA issues may permit generation of huge multimillion patient databases to tailor normal standards and grades of abnormal based on any demographic: age, gender, race, country of origin, weight, height, body size, lean body mass, or any relevant clinical condition (DM, HTN, PAD, cerebrovascular disease, OSA, etc.). Data sets may be immediately and anonymously (HIPAA standards) databased. Clinicians may have their questions better answered and guaranteed that unrecognized relevant coexisting and unrecognized conditions are identified and analyzed. The greater accuracy, efficiency, productivity, and real-time analytics may free up time for more teaching, learning, training, and research.

The disclosed system further provides workflow efficiencies that reduce repetitive tasks and cognitive load on physicians and sonographers. For example, by automating routine analyses and streamlining data presentation, the smart ECHO system 100 reduces potential for human error resulting from operator fatigue. In addition, by providing clear, consistent, and high-quality diagnostic outputs, the system facilitates improved engagement and fosters quality improvement across all members of the clinical team, thereby empowering team members to consider new ideas. New ideas can proceed from theory to practice, more easily developed and tested and incorporated if proven to be best practice.

AI may also improve consistent use of best practices for the most demanding ECHO methods and techniques of 3D/4D, pixel tracking, tissue Doppler imaging, color m-mode and shape specific 2D/3D PISA imaging for valve stenosis regurgitation and shunts. Collective display and analytics of related images may improve awareness and communication.

Current flat screen displays limit display precluding display of multiple, dimension appropriate image display and relevant analytics/links to support/staff. At least some embodiments of the systems described herein incorporated a virtual reality and/or augmented reality display. Such displays may permit increased flexibility and minimize cost. Systems may be able to display the current image, prior study images, measurements, changes (differential/velocity and acceleration of change mapping), the model of heart function and disease, 3D data sets in 3D, true volumetric 3D measurements, and trigger the sonographer and machine to investigate for associated findings, refine imaging logistics, resolve gaps in study, show conflicts, display all associated conditions, adapt to imaging obstacles, and communicate all relevant analytics.

The smart ECHO system 100 allows diseases to be seen in new dimensions. The full dimensional extent, severity, and complexity of disease may lead to disease identification at more treatable stage and improved utilization and timing of interventions. The greater display capability of virtual reality may be best for the supervision echocardiographer(s) and augmented reality display best for transthoracic (TTE) imaging by the sonographer and transesophageal ECHO (TEE) by the echocardiographer.

In the exemplary embodiment, the smart ECHO system 100 uses an ECHO server as the database for image analytics and smart ECHO AI/analytics. Studies read to have proper measurements and methods imbedded in the images according to best practices and all validation methods with disease analytics, data analytics, dimensional analytics, and comparative analytics. These studies may function as the data sets for the smart ECHO image analytics and processing analytics and the other studies ready by current practice methods may serve as the data sets to prefect smart ECHO reprocessing of legacy ECHOs.

HIPAA standardized de-identification of all data sets is done when ECHO studies are transferred to the computer science server/database. The smart ECHO system 100 may perform the image analytics and image pattern analysis; ML and DL of best practice validated measurement techniques; hardware modification; AR integration, and display development with analytics; ML and DL of dimensional and comparative analytics; network development; imbedding disease, predictive, and association analytics; communication method and pathway development and refinement; appropriate use criteria (AUC) based intelligent ordering; HIS connectivity; links between staff; and imbedding and linking relevant and related imaging and report.

ECHO companies/reporting systems have previously followed simple/rigid/user unfriendly/very limited stress options rather the goal/need of complete/flexible/complete intelligent ECHO with real-time data and conflict feedback. Reading station analytics remain primitive and simple. Cross validation methods and validation analytics have never been considered. Diagnostic and interventional TEE have the same issues. The smart ECHO system 100 again is the ideal solution for the same reason. Smart ECHO partnership with TEE could dramatically enhance screening, optimize intraprocedural direction of intervention, and provide immediate and long-term feedback regarding effects of intervention.

The smart ECHO system 100 is based on the highest quality intelligent image acquisition, front end analytics, display technology appropriate for the image type, multidirectional networking, predictive/association analytics, speed of communication and new comparative and dimensional analytics. Data is validated by the principles of conservation of mass, structural data validates functional/Doppler data and the reverse, mechanics, flow dynamics and hemodynamics. Screening smaller more light weight imaging systems is developed with augmented reality (AR) display for limitless display and analytics. All AI and ML/DL for imaging and processing based on legacy and prospective studies are learned according to these validation principles. Servo-intelligent image acquisition is a partnership between the sonographer and smart ECHO. The AI/ML/DL with association and complex predictive analytics is designed to be robust and handle any disease condition, complexity, or severity. Automated databasing and analytics also are imbedded for continuous quality improvement of methods, protocols, image acquisition, processing, data analytics, comparative analytics, and disease analytics.

The smart ECHO system 100 enables hospitals and systems to provide high quality, valid, and reliable diagnostic and screening aligned with best practices without the significant cost associated with additional personnel. By developing a system whereby one sonographer can receive real-time feedback, cross-validate measurements, and reduce errors, the smart ECHO system 100 allows for the highest quality imaging, workplace quality, lifestyle quality, productivity, and time efficiency regardless of location. Work at home may be minimized to prevent burnout. Currently, only the most well-funded hospital systems can afford the time and personnel associated with best practice echocardiography, meaning most patients across the United States receive sub-standard care. The smart ECHO system 100 technology may reduce errors/gaps and erroneous/incomplete reporting at even faster workflow increasing quality of care and equalizing access for patients.

Smart ECHO validation is done with a series of more than 6,000-10,000 ECHOs manually interpreted by an expert echocardiographer with imbedded best practice image recognition, measurements; data, disease, dimensional and comparative analytics. All are cross validated by the laws of conservation of mass, mechanics, flow dynamics, and pressure volume flow and new structure volume flow relationships. The smart ECHO system 100 with computer science and comparative analytics includes the HIPAA compliant archive of ECHOs, data mining of the studies, de-identification of the data, appropriately link new smart ECHO processing/data for serial analytics needed to develop best practice image recognition and structural functional analytics and validation by robust matrices of interdependence and cross validation principles. Manual data/results may be HIPPA mined into large database along with re-interpretation results by AI/ML/DL image, disease, dimensional and comparative analytics. Studies may be processed in groups of 100 with complex database analytics to identify and correct image recognition, analytic and grading errors, and conflicts. The process guarantees validation and accuracy of all analytics.

In one example embodiment, the integrative smart ECHO system 100 between cardiology and computer science/engineering results in: a) 15,000-20,000 patient robust database of for smart ECHO development/validation and data/disease analytics/legacy analytics/comparative analytics of resting TTE, congenital ECHO, hemodynamic ECHO, stress ECHO, and diagnostic and interventional TEE7, b) 2,500 patient database of stress ECHOs/interventional ECHOs for dimensional analytics, c) Prove that quantitative criteria based is better than the current standards for disease screening, disease extent, disease severity, direction of therapy, selection of intervention, timing of intervention, prognosis and outcomes, d) Development of the information network and feedback systems at all stages of the smart ECHO system 100, e) Development of the hardware/software for smart ECHO machines. VR/AR/screenless ECHO machines, f) IT and smart ECHO lab network/hospital network/hospital system network/statewide/national/international HIPAA compliant and secure (anonymously identified) cloud based/other modality databases standards/methods/best practices/protocol standardization/processing/standardization/reporting standardization and research and development. Standards of normal and disease severity to be known for all demographic profile or systemic diseases), g) Smart ECHO continuous quality improvement, h) Smart ECHO system 100/machine/deep learning/artificial intelligence of the process of volume validated m-mode/2D/3D/4D/PW/CW/Doppler methods and measurements/feedback loop for updating protocols/automation of state-of-the-art measurements and best practices/high risk findings/morphology/feedback loops for learning and updating standards for normal and severities of abnormal, i) Feedback loops for identification of data conflicts, alerts for high risk, indications for advanced imaging methods, indications for contrast, myocardial perfusion imaging, enhanced use of contrast imaging and multi-modality ECHO imaging, j) Smart ECHO for intelligent reprocessing/image analysis and automated front end comparative analytics with digital acquired ECHOs with only the current standard qualitative analytics and AI for image recognition/analysis/processing, k) Complete hemodynamic, structural functional profile and AI feedback and databasing, AI validation of all studies/databasing of all studies/constant QA of protocols/constant updating of standards for normal and disease severity, and protocol for selection of data to be sent to providers, l) Development of a centralized imaging database for all cardiac imaging (ECHO/MRI/CT/Nuclear) with maximized data extraction/hemodynamic profiling for inter-modality comparison, and constant updating of the unique hemodynamic/cardiac profile of the patient irrespective of the specific modality (standardization of the data sets for all cardiac imaging modalities). m) Training of sonographers/working with sonographer schools, n) Training of intermediate imagers/fellows, o) Training of echocardiographers, p) Training in new approaches to research. q) System/IT development for seamless uploading of reports and data to hospital network with prioritized/timely reporting/prioritized communication with providers, r) Constant QA for staff/sonographers/trainees/echocardiographers, s) System learning to minimized wasted time, t) System learning for front end AUC ordering and back-end confirmation, such as Improved utilization of technologies, expansion to avoid missing high risk patients and minimization of redundancy and waste, u) AI for protocols that further minimization of nondiagnostic studies. The end of the incomplete study, and v) AI/system and machine/deep learning based on in house and national/international data to constantly maintain sonographers and echocardiographers at state of the art/best practice level of understanding/training level/train trainees to achieve the same.

At least some embodiments of the smart ECHO system 100 described herein utilize a three step process of acquisition and data processing (intermediate and final reporting) founded on the principles that (1) Doppler valve stroke volume data validate 2D/3D/4D chamber volume data, (2) 2D/3D/4D chamber volume data validate Doppler valve stroke volume data, (3) accuracy of multiple levels of derived data/calculations dependent on accuracy primary data, (4) left and right heart always interdependent, (5) volume validated imaging data are the foundation for accurate assessment of site specific pathology, (6) conservation of mass and the laws of physics and hemodynamics are the foundation for primary data validation, and (7) enhanced assessment of anatomy by automation of all measures of severity of anatomic disorders on all studies by machine and deep learning (better definition of normal and disease/severity). The smart ECHO may enhance/guarantee the quality of the volume data by directing the sonographer to acquire the data at the identical guideline directed position. For A Fib/arrhythmias and all patients, anatomic and Doppler data may be measured at the same RR interval/same number of cycles to endure similar loading conditions. All abnormalities described and investigated by multidimensional (multiple measurements and calculations) approach and all abnormalities quantitated by consensus of multiple methods of quantitation. Elements of the model of heart function may be available to sonographer in real time so data conflicts automatically trigger resolution/mechanism investigation/specialized imaging. Ensured validity/more complete description of the disease state and better prognostic information should lead to more confident, accurate and immediate multidimensional (time, condition, and intervention) awareness of disease extent. The switch from non-anatomic to true anatomic, physiologic, pathophysiologic, and severity data may permit reliable and more valued inter-modality comparison of ECHO data with CT/MR/SPECT/PET.

The net effect of ECHO modeling by a complex matrix of mathematics (matrix algebra, geometry, trigonometry, calculus, differential equations) and the physical principles of ultrasound, Doppler, flow dynamics, mechanics, conservation of mass, and pressure-volume-flow and structure-volume-flow relationships may be better definition of normal, normalcy and grading of disease state and extent. The system provides multidimensional intelligence to the sonographer at the front end/enhanced training of intermediate staff/trainees. Disease specific data may be more accurate and based on a guaranteed accurate primary data with the complete and reliable/precise hemodynamic profile.

Secondary/tertiary/quaternary/calculated anatomic and hemodynamic data may also be based on improved accuracy of primary data. All studies may have complete data sets/hemodynamic profiles/accuracy for learning, databasing and filtering reports to the needs of providers or specialists.

At least one aspect of the present disclosure that enables the smart ECHO system 100 to achieve necessary quality standards for AI/ML/DL ECHO imaging and automated report generation is the rigorous validation process proposed against the gold standard of the database/archive of ECHOs manually measured and interpreted with all the validation analytics. This archive provides a true gold standard and best practice ECHO interpretation because all of the studies have been validated with clinical, angiography, invasive hemodynamic and other imaging studies. The analytics are developed and refined based on the entire ECHO study. The system may further be advanced by building in potential/expected findings to be triggered by ordered indication (intelligent probability), clinical diseases, patient status (stable/critical/shock/arrest/severe HTN, etc.), and expected associated findings based on any moderate or severe finding. Thereby, prior knowledge/models of disease can be used to enhance the probability of detecting the true extent, scope, and complexity of the abnormalities and disease. Embodiments of the present disclosure utilize a feedback loop process of individual study and group database/analytic assessment of visual analytics, image extraction analytics, ML and DL analytics, image and Doppler measurement analytics, structural recognition analytics, disease management analytics, data driven reporting analytics, comparative analytics, and dimensional analytics in groups of 100 studies. In certain implementations of the model development process, the smart ECHO system may undergo iterative training sessions on at least a weekly basis. By approximately the fiftieth training cycle, the system may have substantially reduced or resolved the majority of detected errors and conflicts, thereby converging toward a validated and optimized configuration of AI, ML, DL, and associated analytical models.

Database development, data mining, data analytics and appropriate statistics may be used to confirm that the smart ECHO system 100 is correctly analyzing and processing images, performing measurements/calculations with correct analytic cross validation by comparative analytics with the ECHOs best practice measured/validated and processed/interpreted by one or more experienced and/or expert cardiographers. Groups of 100 ECHOs may be processed by image AI/ML/DL methods and comparative analytics done for conflict/error identification and correction. Every study may be directly compared to the manual interpretation. Specific disease states may also be compared to identify and correct any analytic errors. Disease grades, normal values, relevant links/associations between findings may also be compared to identify and correct any errors.

In certain embodiments, the smart ECHO system 100 is configured to perform structure and plane recognition with an error tolerance near or approaching zero, such that misidentification or omission of anatomical structures is minimized or eliminated. The smart ECHO system 100 may be programmed such that near or all grades for pericardial and aortic pathology are correlated and congenital diseases are correctly identified. Conflicts with conservation of mass, mechanics, flow dynamics and hemodynamics are identified and resolved by the methods of cross validation. In some embodiments, grades for thickness, calcification, mobility, pathology, may have variability less than 0.5% grade. Example tolerance levels may include 2-3% for caliper measurements, 3-5% for area measurements, and 5% for volume measures and higher-level calculations. Measurements can be done according to RR intervals (e.g., the duration between the peak of one R wave and the peak of the next R wave on an electrocardiogram (ECG)) reported in the manually interpreted studies.

The following measures may undergo comparative analytics of the AI/smart ECHO methods with the gold standard of each unique ECHO profile and include chamber areas, dimensions, volumes ejections fraction, longitudinal/circumferential and strain, strain rate, wall thickening, wall motion, SAX area changes, mass, fractional shortening, wall motion, scar extent/burden, and timing/pattern, specking, tissue characteristics, myocardial perfusion, color Doppler patterns, continuous and pulsed wave Doppler and velocity time integrals. Stroke volumes for chambers and valves with confirm function. Precise color m-mode of LV inflow propagation. All structures will be assessed with best methods. Any valve stenosis may be precisely graded for severity and complexity by all passible analytics for etiology, morphology and awareness of hemodynamics. All parameters may be indexed to BSA. Any valve regurgitation may be assessed by all possible methods for etiology, morphology, grade and rates of change. Alarming changes may be immediately identified. Each important finding may be confirmed by all possible analytics and measures of structure.

After final validation of analytics, the smart ECHO system 100 may function as a foundation of legacy analytics of archived ECHOs. The smart ECHO system 100 may ensure all legacy studies are up to speed with current practice methods of image and data analytics to identify conflicts; unrecognized measurement, processing, grading, reporting errors; common errors; beneficial effects of legacy study analytics; potential clinical issues/implications relating to legacy analytics; disease and overall magnitude/value of corrections and enhanced analytics.

Workflow may be monitored for completeness, length and time efficiency of studies, time from ordering to study performance, time from ordering to final reporting, time from reporting to communication with providers, efficiency of linking clinical information and efficiency of linking relevant imaging.

For left sided regurgitant lesions, the stroke volume of the regurgitant valve equals the LV stroke volume and is greater than the other valve by the regurgitant volume. For lesions of both valves, the LV stroke volume is equal the stroke volume of the valve with the largest regurgitant volume plus the smaller regurgitant volume of the second valve. The stroke volume across each regurgitant valve (aortic and/or mitral) is the stroke volume of the right heart valve with no or minimal regurgitation (usually the RVOT and transpulmonary stroke volume) with the regurgitant volume of the left sided valve. Volume data can then be corroborated by state-of-the-art PISA derived volumes.

For shunts, the stroke volumes of the of the sending and receiving chambers plus circuit involved chambers/valves is the same (as long as there are no regurgitant valves; if valve regurgitation is present, the involved chambers and valve stroke volume is larger by the regurgitant volume) and larger than uninvolved chambers valves by the shunt volume (as long as there are no regurgitant valves, if present the involved chambers and valve stroke volume is larger by the regurgitant volume).

For right sided regurgitant lesions, the stroke volume of the regurgitant valve equals the RV stroke volume and is greater than the other valve by the regurgitant volume. For lesions of both valves, the RV stroke volume is equal the stroke volume of the valve with the largest regurgitant volume plus the smaller regurgitant volume of the second valve. The stroke volume across each regurgitant valve (tricuspid and/or pulmonic) is the stroke volume of the left heart valve with no or minimal regurgitation (usually the LVOT and transaortic stroke volume) with the regurgitant volume of the right sided valve. Volume data can then be corroborated by state-of-the-art PISA derived volumes.

For stenotic valves without insufficiency, the stroke volume of the inflow to the valve and any valve with no/minimal insufficiency is equal the stroke volume at the valve tips (narrowest site, continuity equation). Velocity and VTI ratios can be used to define data with correlation to the 2D/3D valve size by planimetry. Accuracy can be maximized by 2D/3D inflow areas by direct planimetry/at exact level of pulse Doppler sample. PISA method can also corroborate maximum area (monophasic valve opening) and mean area (multiphasic valve opening). The increased precision and constellation of data can be used to validate resistance and impedance data for enhanced valve/disease/pathophysiologic/prognostic assessment.

For stenotic valves with significant insufficiency, the stroke volume of the inflow to that valve only (guaranteed conservation of mass) is equal the stroke volume at the valve tips (narrowest site, continuity equation). Velocity and VTI ratios can be used to define data with correlation to the 2D/3D valve size by planimetry. Accuracy can be maximized by 2D/3D inflow areas by direct planimetry/at exact level of pulse Doppler sample. PISA method can also corroborate maximum area (monophasic valve opening) and mean area (multiphasic valve opening). The increased precision and constellation of data can be used to validate resistance and impedance data for enhanced valve/disease/pathophysiologic/prognostic assessment.

For aortic mechanical prosthetic valves (LVOT) and aortic (LVOT) and mitral bioprosthetic/ring/band repaired valves (valve annulus or ring plane) with no or minimal insufficiency, the stroke volume of the inflow to the valve and any valve with no/minimal insufficiency equals the stroke volume at the valve tips (narrowest site, continuity equation). For mitral mechanical valves with no or minimal insufficiency, the stroke volume of any native valve with no/minimal insufficiency equals the stroke volume at the valve tips (narrowest site, continuity equation). Velocity and VTI ratios can be used to define data with correlation to the 2D/3D valve size by planimetry. Accuracy can be maximized by 2D/3D inflow areas by direct planimetry/at exact level of pulse Doppler sample. PISA method can also corroborate maximum area (monophasic valve opening) and mean area (multiphasic valve opening). The increased precision and constellation of data can be used to validate resistance and impedance data for enhanced valve/disease/pathophysiologic/prognostic assessment.

For aortic mechanical prosthetic valves (LVOT) and aortic (LVOT) and mitral bioprosthetic/ring/band repaired valves (valve annulus or ring plane) with significant insufficiency, the stroke volume of the inflow to that valve only equals the stroke volume at the valve tips (narrowest site, continuity equation). For mitral mechanical valves with no or minimal insufficiency, the stroke volume of any native valve with no/minimal insufficiency plus the regurgitant volume of the prosthetic valve equals the stroke volume at the valve tips (narrowest site, continuity equation). Velocity and VTI ratios can be used to define data with correlation to the 2D/3D valve size by planimetry. Accuracy can be maximized by 2D/3D inflow areas by direct planimetry/at exact level of pulse Doppler sample. PISA method can also corroborate maximum area (monophasic valve opening) and mean area (multiphasic valve opening). The increased precision and constellation of data can be used to validate resistance and impedance data for enhanced valve/disease/pathophysiologic/prognostic assessment.

2D/3D mitral/aortic/tricuspid/pulmonic valve directly measured size data validate Doppler/volume data and further ensure the accuracy of the Doppler/physiologic data sets for interpretation, disease extent/severity/complexity of disease/number of disease states, deviation from normal and continuous updating of normal/grades of abnormal relative to any demographic parameter(s).

Anatomic valve size and Doppler stroke volume data and global chamber function data validate 2D/3D LV/RV chamber systolic and diastolic volumes and generate new data sets for normal/severity of abnormal. Furthermore, the high confidence and accuracy of the volume data can cross modalities and ensure all modalities (nuclear, CT, MRI) are complementary and directly comparative with no fudge factors. The true/reproducible volumes and hemodynamic profiles and data sets should finally be simultaneously and universally discoverable. Obstacles to intra- and inter-modality comparative analysis may be resolved.

Valve morphology/anatomic/structural data to be to be correlated with all pathophysiologic data to better define disease state, condition, and therapeutic/management/intervention options. Valve and chamber morphology to be checked and confirmed by conservation of mass data so mechanisms of dysfunction induced in reports for follow and management.

In addition to 3D, TDI, PISA, pixel tracking and 2D longitudinal/radial/circumferential strain, myocardial perfusion may be quantitated in a novel process according to consensus protocols, such as the Porter/Wei/Kaul. The consensus protocols may be continuously updated in response in changes to consensus protocol and/or to account for timing/completeness/intensity/pattern and capillary integrity/capillary perfusion/myocardial viability/contractile reserve. Regional maps can simultaneously correlate perfusion data with thickness/strain for higher fidelity viability/contractile potential/ischemic assessment.

Intracardiac and selected extracardiac shunts (ASD/VSD/PDA) may be universally standardized and validated by the PISA and volume methods. Automated advanced 2D/3D shape analysis and multiphasic (systolic and diastolic shunt orifice size/phase related and total shunt volume and shunt fraction/pulmonary to systemic flow [Qp/Qs] ratio). Validation again by volumetric Qp/Qs analysis (total volume by conservation of mass principle).

The primary function of the heart is blood flow and circulation and these functions form a foundation of ECHO image and data analytics. Conservation of mass principle may link anatomy, myocardial perfusion, pathophysiology, enhanced definitive assessment of hypertrophic obstructive cardiomyopathy, sub-valvular and supra-valvular stenoses/major complications/shunts related cardiac open surgical, minimally invasive, percutaneous and transcatheter interventions (type of intervention, mechanical/bioprosthetic prosthetic, remodeling surgery, myectomy, structural, congenital, plugging, device/shunt closures, etc.).

The smart ECHO system 100 may achieve servo-interactive machine-sonographer (ECHO technician) and machine-sonographer-echocardiographer (trans-esophageal TEE and interventional TEE or transthoracic TTE ECHOs) intelligent image acquisition and real-time processing/display of results. The system is modified for limitless augmented reality display of any and all analytics and correct display of images regardless of dimension and correct image processing in the correct dimensional display.

The smart ECHO system interacts with the sonographer via audio or visual for coaching intelligent image acquisition with correct machine settings and correct planes and cursor positions. All studies may be complete with no gaps or missing/erroneous images or data/data analytics.

The smart ECHO system 100 may evolve to state-of-the-art AR technology and system with the software and modified hardware to adapt to the state of the art ECHO machine; build and adapt the display system to handle modeling, image display according to dimensions, dimensions analytics, new disease state analytics, comparative analytics; imbed the AI/software/boards/display in the machine; and modify the machine to a more compact and flexible system capable other displays in the hospital.

The smart ECHO system 100 includes an intelligent multidimensional network with AR for all members of team and AR interactive display communication. This may allow members of ECHO lab to wear AR systems/helmets/glasses. Current hospital networks and local networks have very simple back end interaction, often requiring a network with robust real-time front-end interaction and communication. smart ECHO analytics can be applied to legacy ECHO not processed by legacy methods so every dimensional ECHO is processed with maximum quality and state of the art analytics. Images and processing grades and data may be upfront displayed for the smart ECHO system and sonographer for real-time image and data analytics and change display/comparative analytics (never been done but could game change quality). All pre-existing conditions can be correctly investigated/comparatively graded.

The smart ECHO includes live AR displays capable of all smart ECHO displays for sonographers (cardiology staff/faculty), fellows, nurses (more limited display focused on patents) stress technicians, and support staff. Some embodiments may include seamless servo-links (video, audio, etic) developed from the smart ECHO machine and sonographer to the echocardiographer, fellows and support staff. Appropriate dimensional (rhythm, BP, stress, intervention, heart rate, or clinical condition change) analytics can then be added for the goal of final highest quality reporting as patient leaves the ECHO suite and the sonographer leaves the room or portables.

The smart ECHO system 100 includes state-of-the-art multi-dimensional/multidirectional network and AR displays for all lab and cardiology technical and professional to develop the seamless multidimensional display/communication/reporting to make the fastest workflow and highest quality ECHO/Imaging labs imaginable and technically possible.

The smart ECHO system 100 networks with providers/other cardiac labs and to hospital network. The goal is seamless almost immediate communication of highest quality results to providers and feedback. The smart ECHO system 100 includes communication with other cardiac labs and operating rooms via AR headsets and with ordering providers, such as via a multi-dimensional/-directional network.

The smart ECHO system 100 may include correct appropriate use criteria (AUC) for all studies with links to relevant clinical information and all legacy imaging (ECHOs, MR, CT, X ray, nuclear) for report and display analytics by the smart ECHO system 100 as the studies are done. The ECHOs are not analyzed solely based on ECHO knowledge, but instead, the ECHOs are performed and analyzed/processed in the global clinical setting with awareness and comparative analytics to all related images.

The smart ECHO system 100 is based on data mining of 15,000-20,000 TEE, stress, intervention, and TTE ECHOs with all studies de-identified according to HIPPA standards with automated/permanent obscuring of patient name and medical record number. Each ECHO is identified by a new database number and reports data measured is also de-identified and linked to the new database number. All clinical data for protocol development is de-identified and linked to the database number. Each additional ECHO with serve as a learning opportunity for smart ECHO to improve and evolve.

All the studies are read by the system and subject matter experts according to best practice methods of data validation and conflict resolution by the laws of physics/hemodynamics with comprehensive structural and functional modeling of cardiac function. The analytics and application of smart ECHO analytics also validate and perfect the legacy analytics. Followed by careful analytics to assess the effects of legacy analytics on ECHO and quality/patient care management.

The images and some prospective studies are used with imbedded measures for AI/ML/DL to learn the planes of ECHO, correct and improper machine settings based on best practices, recognition of structure, description of structure, learn how to correctly measure pictures and movies of structure and Doppler information, learn all the measures and calculation, learn the interdependence of data, learn how to cross validate according to laws of physics, learn how structure validates Doppler and Doppler validates structural data, learn associated findings to be expected based on disease and severity, learn how structural information interacts with Doppler to provide physiologic/functional/hemodynamic information, learn how to grade findings/disease conditions for severity/complexity/extent/interdependence.

The smart ECHO system 100 teaches complete reporting; modeling of heart structure, chamber volumes, chamber mass, chamber function/physiology/pressures/gradients, valve function, and associated pericardial and aortic findings; derive disease(s) type and grade/extent and then learn data driven reporting.

The smart ECHO system 100 includes a robust database for data analytics and derive normal values and grades of disease according to these properly validated methods. Publications to support patenting of the computer science methods and smart ECHO profiling of the heart. This includes the process of dimensional and comparative analytics for stress/interventional studies and legacy studies.

The smart ECHO system 100 uniquely includes servo-interactive machine-sonographer (ECHO technician) and -echocardiographer (trans-esophageal TEE and interventional TEE or transthoracic TTE ECHOs) intelligent image acquisition and real-time processing/display of results. Current systems use only a flat screen display which precludes sophisticated display of data/data analytics, correct display of 3D and 4D images, group display of associated images and Doppler data, method of conflict resolution and direct comparative analytics/display of changes. The smart ECHO system 100 includes limitless augmented reality display of any and all analytics and correct display of images regardless of dimension and correct image processing in the correct dimensional display.

The smart ECHO system 100 interacts with the sonographer via audio or visual for coaching intelligent image acquisition with correct machine settings and correct planes and cursor positions. All studies are complete with no gaps or missing images or data/data analytics. Abnormalities may be identified in real-time, and trigger best practice complex analysis for group display of all images related to that structure or functional abnormality. Conflicting data/results may be real-time identified for resolution by artifact correction or disease/pathology. Appropriate uses for contrast and bubble ECHOs may be imbedded for image and Doppler enhancement, myocardial perfusion, and shunts according to findings and disease. Expected associated findings and physiology based on disease(s) type and severity may also be real-time displayed to the sonographer and the complete data driven report may be displayed at completion of the study to the sonographer with all appropriate data analytics, predictive analytics, dimensional analytics, and comparative analytics to make sure the report fits the sonographer and smart ECHO impressions of the patient and conditions.

The smart ECHO system 100 includes a robust database for data analytics and derive normal values and grades of disease according to these properly validated methods. Publications to support patenting of the computer science methods and smart ECHO high flexibility and dimensionally appropriated AR display of imaging and analytics. The smart ECHO system 100 includes dimensional and comparative analytics for stress/interventional studies and legacy studies.

The smart ECHO system 100 includes intelligent multidimensional network with AR for all members of team and AR interactive display communication. All members of ECHO lab may wear AR systems/helmets/glasses. Current hospital networks and local networks have very simple back end interaction. The smart ECHO system 100 includes a new network with robust real-time front-end interaction and communication. smart ECHO analytics are applied to legacy ECHO not processed by smart ECHO methods so all are processed with maximum quality and state of the art analytics. Images and processing grades and data may be upfront displayed for the smart ECHO and sonographer for real-time image and data analytics and change display/comparative analytics (never been done but could game change quality) so all pre-existing conditions are correctly investigated/comparatively graded.

Live AR displays capable of all smart ECHO displays for sonographers (cardiology staff/faculty), fellows, nurses (more limited display focused on patents) stress technicians, and support staff. Seamless servo-links (video, audio, etic) from the smart ECHO machine and sonographer to the echocardiographer, fellows and support staff. Rel-time identification and communication of high-risk conditions. Real-time display of the results or final display or report/images after approval by sonography/smart ECHO may be communicated to the fellow and echocardiography for final quality control of the data/real-time group display of findings, resolved conflicts/data validation/final analytics and comparative visual and reporting data analytics. Appropriate dimensional (rhythm, BP, stress, intervention, heart rate, or clinical condition change) can then be added with the goal of final highest quality reporting as patient leaves the ECHO suite or smart ECHO/sonographer leave the room or potables.

The database may also derive normal values and grades of disease according to these properly validated methods. Publications to support patenting of the computer science methods and smart ECHO network/legacy analytics. The AI/ML/DL includes dimensional and comparative analytics for stress/interventional studies and legacy studies. Database development, expansion, mining and data analytics include the process of a) standards for normalcy and grades of disease severity, extent and complexity; b) dimensional and comparative analytics for stress/interventional studies and legacy studies, c) new markers of risk and prognosis; d) development and refinement of structural, functional, and hemodynamic modeling of disease and heart function; e) dimensional and comparative analytics for stress/interventional studies and legacy studies/showing the add valve relative to current qualitative reading standards; and f) disease description with new dimensional analytics only possible by automated AI/ML/DL intelligent acquisition and analytics.

The smart ECHO system 100 is configured to network with providers/other cardiac labs and to hospital network. The goal is seamless almost immediate communication of the highest quality results with the providers. All possible types of communication with other cardiac labs and operating rooms via AR headsets and with ordering providers. The complete report with imbedded appropriate picture or video examples of pathology may need to be real time displayed for operator awareness and the goal of tailored and maximum effectiveness of communication for maximum provider impact/utilization.

The smart ECHO network/lab interacts with the cardiology technical and professional to develop the seamless multidimensional display/communication/reporting to make the fastest workflow and highest quality bidirectional communication with information system providers/staff and intelligent ordering to link with all appropriate clinical information/imaging and best practice AUC based ordering.

Some embodiments of the present disclosure utilize a complexity analysis for reporting including one or more of: a) Investigation for all typical findings associated with moderate to severe findings, b) Severity standards drive reporting, c) Continuous AI/machine learning and deep learning of typical disease profiles and possible findings exacerbating patient condition, d) Change differential mapping of disease/condition/hemodynamic profiles/anatomic information, e) Relevant reporting and awareness to imagers of disease acceleration rather than linear velocity change, f) High risk conditions identified and reported to sonographer and echocardiographer/intermediate staff, g) Immediate order sets uploaded when additional noninvasive or invasive imaging needed, and h) Immediate communication of high risk to ordering provider and uploading of order sets for surgical consultation(s).

Some embodiments of the present disclosure utilize intelligent modeling that includes one or more of: a) Moderate and severe abnormalities as triggers, b) Appropriate specialized testing, c) Awareness of common associations, d) All pre-existing findings verified, e) Common associated anatomic and physiologic/pathophysiologic abnormalities and grades of findings, f) Investigation for possible findings based on clinical changes, stress and interventions, g) Moderate to severe anatomic abnormalities to trigger investigation of Doppler/hemodynamic disturbances, h) Doppler abnormalities and type to trigger investigation for anatomic abnormalities, i) Heart rhythm abnormalities to trigger investigation for cases, j) Interventions to trigger investigation for possible volumetric and functional changes (wall motion, strain, stress, valve function, systolic and diastolic function, resistance, compliance and pressures/gradients, k) Interventional methodologies to trigger investigation for expected findings/investigations (trans-septal to trigger screen for shunts and ASDs, Mitra clips trigger compete volumetric and functional assessment/valve stenosis screening, plugging PVL/VSD/fistulas/TAVR/TMVR appropriate screen, and l) Sx with nondiagnostic/negative studies to trigger dimensional analysis/stress/provocative testing.

Some embodiments of the present disclosure utilize a matrix of interdependence that includes one or more of: a) All valves and chambers linked by volumes and flows, b) Anatomy linked by volumes and flows, c) Resistance only accurate when volume and flow correct, d) Anatomy validates Doppler/physiology, e) Doppler/physiology validates anatomy, f) LV and RV stroke volumes the same except with beat to beat variation in constriction/overall minute volumes the same, g) Dynamic flow associated with mean areas and laminar flow with max single valve areas, h) Pressures, flows, resistance volumes always linked, i) Stress directly related to pressure and size and inversely related to thickness, j) Velocity, pressure linearly related to flow at low resistance and geometrically related at high resistance, k) Time integrals of velocity reflect flow, l) Gradients related to flow, m) Basal volume of flow in and out of heart reflected Doppler and anatomy of all valves and chambers, n) Pressure development related to stiffness, relaxation, scarring, dyssynchrony, timing of events, restrictions, o) Valve function related to impendence, pressure, vascular/chamber stiffness/resistance, intrinsic properties and anatomy of the valve, and p) Chamber contraction related to pressure, wall stress, strain, muscle mass, calcium, hormones, support structure/infrastructure.

Some embodiments of the present disclosure utilize principles of conservation of mass that includes one or more of: a) Flow entering the heart equals the flow exiting the heart, b) Continuity equation, c) The concept connecting anatomy and physiology, d) Error and conflicts due to measurements at different sites, e) Resolution by special memory and awareness, f) Valve size validated by volume/flow data (annulus and outflow), g) Shunts, h) Valve stenosis, i) Aneurysms, j) Valve regurgitation, k) Fistulas, l) Relevant factors for validation of valve size, m) Relevant factors for validation of diastolic function, n) Relevant factors for validation of valve resistance and chamber impedance, o) Relevant factors for validation of pulmonary and systemic vascular resistance, and p) Relevant factors for validation of chamber volumes and systolic function.

Some embodiments of the present disclosure utilize best practice development and incorporation that includes one or more of: a) Machine settings optimized, b) Protocols optimized, c) Auto-feedback to practices of sonographer, d) Best practice use of specialized testing, e) Best practice use of contrast for 2D/3D imaging, f) Best practice use of strain imaging, g) Best practice use of X plane imaging, h) Best practice use of multiplane imaging, i) Best practice use of multiplane color Doppler imaging, j) Best practice use of 3D/4D/2D/m-mode imaging, k) Best practice use of spectral Doppler imaging, l) Best practice use of spatial memory for combining anatomic and Doppler imaging, m) Best practice use of dimensional imaging, n) Optimized gain, compression, depth, magnification, sweep, scale, baseline, gate size and filter settings for each Doppler image and plane, o) Optimized use of contrast for Doppler imaging of valve stenosis and regurgitation (quantitation), p) Best practice use of extended imaging (aorta) based on clinical history, q) Best practice use of anatomic and pathophysiologic imaging for commonly associated findings (disease extent and complexity) with any moderate to severe findings, and r) Best practice comparative imaging and analysis for legacy/serial and dimensional studies to ensure complementary data analytics/enhanced information, risk assessment, guarantee appropriateness, enhanced modeling, change/differential/acceleration mapping, risk assessment and avoid redundant imaging.

The smart ECHO Concept: Machine learning/deep learning/data analytic/predictive analytics/comparative analytics of methods utilizes constant refinement for appropriate evolution.

Some embodiments of the present disclosure utilize regeneration of standards and measures of severity that includes one or more of the following principles: a) Initial standards based on current standards, b) Intelligent information from clinical notes and other assessments/imaging/angiography/catheterizations/hemodynamic data, c) Confirmation or correction of clinical information, d) Each patient/ECHO functions as a new addition to normal values or database of abnormal, e) Any demographic for normal values and disease grades, f) Hospital system and network wide databases (ultimate goal international database), and g) Findings missed on prior images/ECHOs and new associations.

Some embodiments of the present disclosure utilize protocol standardization/refinement/new methods that includes one or more of: a) Complete protocol on complete anatomic and hemodynamic profile, b) Partnership between ECHO machine and sonographer, c) Better practices easily added and incorporated, d) Retrospective analysis of data based studies and prospective analysis of new studies, e) Rapid validation of new measurements and calculations/derivatives, f) Constant refinement of intelligent image acquisition and acquisition protocol, g) Protocols always updated to best practice standardized protocol, and h) All reports tailored to complete hemodynamic profile/anatomic profile and complete validated/reproducible/comparable and standardized/criteria and observation/data driven reporting.

Some embodiments of the present disclosure utilize research innovation that includes one or more of: a) Refinement of acquisition protocol, b) New imaging methods, c) Compensation for motion of heart structures in space, d) Dynamic multiplane imaging, e) Rhythm, interval and condition specific standards and disease dimensions, f) Disease/dysfunction burden bases on rate, rhythm, QRS complex, conduction abnormality, clinical parameter, coexisting disease, potential intervention, prediction of effects of interventions, g) Mean valve areas as new marker of valve function especially AV valves, h) Validation of new methods based on retrospective analysis of legacy studies and prospective analysis of new studies, i) New much larger hospital, system-wide, state-wide, nation-wide, international databases of anatomic and hemodynamic profiles for improved prediction of prognosis/adverse outcome and parameters for screening for/following conditions/diseases/disorders, j) Research on scale never seen before or previously considered (thousands to millions), and k) Easy reprocessing of studies to nearly immediate validate or invalidate hypotheses.

Some embodiments of the present disclosure utilize intelligent associations based on significant findings that includes one or more of: a) Coexisting findings with congenital heart disease/common interventions/repairs, b) Valve disorders (anatomic and disease variants) and structural conditions, c) Clinical conditions/findings/disorders and expected findings based on severity and complexity, d) Clinical decompensation and expectations, e) Rhythm abnormalities and structural/functional findings/abnormalities, f) ECG abnormalities and structural/functional findings/abnormalities, g) CXR findings and structural/functional findings/abnormalities, h) Reliable exam and structural/functional findings/abnormalities, i) Intelligent processing of clinical notes and other tests/imaging/findings, and j) All imaging to be complementary.

The smart ECHO system 100 provides for Refinement of automated intelligence/dimensional analysis (rhythm, status, condition)/data analytics/predictive analytics/comparative analytics. Artificial Intelligence (AI) based two-way network with Augmented Reality (AR) based intelligent acquisition and front-end data analytics/continuous as needed communication between sonographer and echocardiographer.

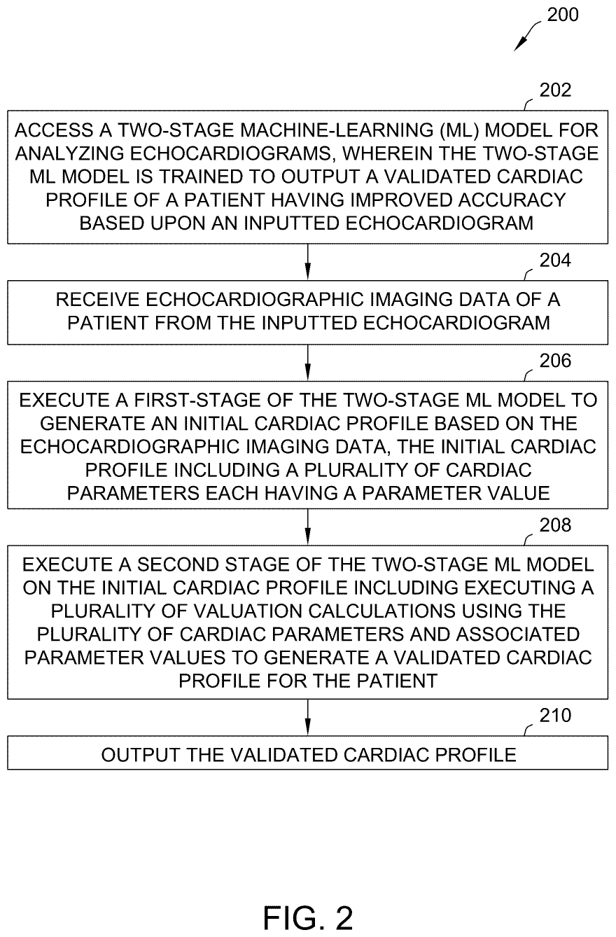

FIG. 2 is a flow chart of a process 200 for analyzing ECHO results using the system shown in FIG. 1. The process 200 includes accessing 202 a two-stage machine-learning (ML) model for analyzing echocardiograms, wherein the two-stage ML model is trained to output a validated cardiac profile of a patient having improved accuracy based upon an inputted echocardiogram. The 200 process further includes receiving 204 echocardiographic imaging data of a patient from the inputted echocardiogram. The 200 process further includes executing 206 a first-stage of the two-stage ML model to generate an initial cardiac profile based on the echocardiographic imaging data. The initial cardiac profile includes a plurality of cardiac parameters each having a parameter value. The 200 process further includes executing 208 a second stage of the two-stage ML model on the initial cardiac profile. Executing the second stage includes executing a plurality of validation calculations using the plurality of cardiac parameters and associated parameter values to generate a validated cardiac profile for the patient. The 200 process further includes outputting 210 the validated cardiac profile.

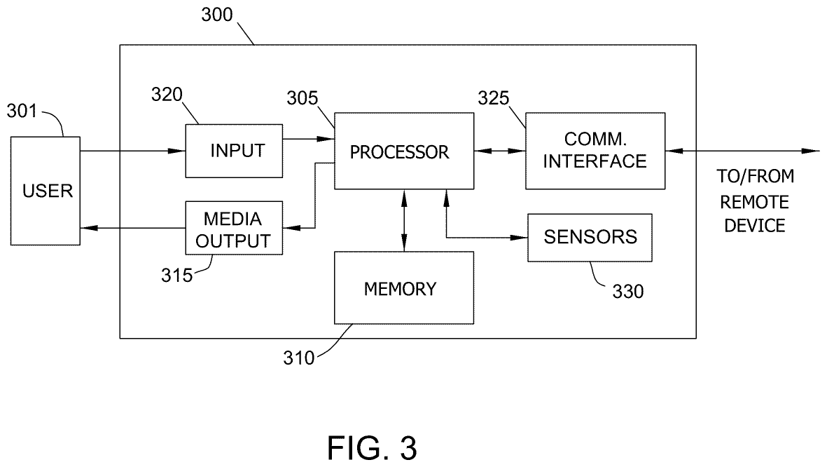

FIG. 3 depicts an exemplary configuration of a user computer device 300 for use with the system 100 shown in FIG. 1, in accordance with one embodiment of the present disclosure. User computer device 302 may be operated by a user 301. User computer device 302 may include, but is not limited to, user device 104 and image server 102 (shown in FIG. 1). User computer device 302 may include a processor 305 for executing instructions. In some embodiments, executable instructions are stored in a memory area 310. Processor 305 may include one or more processing units (e.g., in a multi-core configuration). Memory area 310 may be any device allowing information such as executable instructions and/or transaction data to be stored and retrieved. Memory area 310 may include one or more computer readable media.

User computer device 302 may also include at least one media output component 315 for presenting information to user 301. Media output component 315 may be any component capable of conveying information to user 301. In some embodiments, media output component 315 may include an output adapter (not shown) such as a video adapter and/or an audio adapter. An output adapter may be operatively coupled to processor 305 and operatively couplable to an output device such as a display device (e.g., a cathode ray tube (CRT), liquid crystal display (LCD), light emitting diode (LED) display, or “electronic ink” display), an audio output device (e.g., a speaker or headphones), virtual headsets (e.g., AR (Augmented Reality), VR (Virtual Reality), or XR (extended Reality) headsets), and/or voice or chat bots.

In some embodiments, media output component 315 may be configured to present a graphical user interface (e.g., a web browser and/or a client application) to user 301. A graphical user interface may include, for example, an interface for displaying model outputs, results, inferences, developer actions, etc. In some embodiments, user computer device 302 may include an input device 320 for receiving input from user 301. User 301 may use input device 320 to, without limitation, view model parameters and/or outputs and make changes to ML model 110.

Input device 320 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 315 and input device 320.

User computer device 302 may also include a communication interface 325, communicatively coupled to a remote device such as the server system 108 (shown in FIG. 1) and/or the image server 102 (shown in FIG. 1). Communication interface 325 may include, for example, a wired or wireless network adapter and/or a wireless data transceiver for use with a mobile telecommunications network.

Stored in memory area 310 are, for example, computer readable instructions for providing a user interface to user 301 via media output component 315 and, optionally, receiving and processing input from input device 320. A user interface may include, among other possibilities, a web browser and/or a client application. Web browsers enable users, such as user 301, to display and interact with media and other information typically embedded on a web page or a website from the server system 108. A client application allows user 301 to interact with, for example, server system 108. For example, instructions may be stored by a cloud service, and the output of the execution of the instructions sent to the media output component 315.

Processor 305 executes computer-executable instructions for implementing aspects of the disclosure. In some embodiments, the processor 305 is transformed into a special purpose microprocessor by executing computer-executable instructions or by otherwise being programmed.

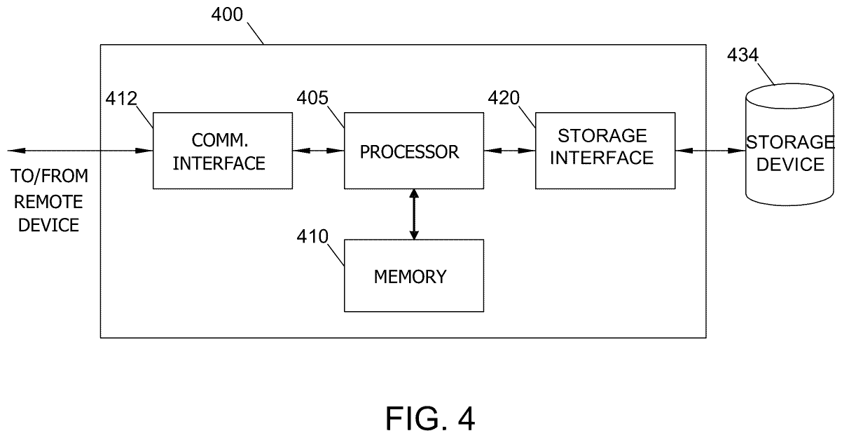

FIG. 4 depicts an exemplary configuration of a server computer device 400 for use with the smart ECHO system 100 (shown in FIG. 1), in accordance with one embodiment of the present disclosure. Server computer device 400 may include, but is not limited to server system 108 (shown in FIG. 1). While only a single server device 400 is shown, in some embodiments, server system 108 of FIG. 1 includes a plurality of the server devices 400 shown in FIG. 4. Server computer device 400 may also include a processor 405 for executing instructions. Instructions may be stored in a memory area 410. Processor 405 may include one or more processing units (e.g., in a multi-core configuration).

Processor 405 may be operatively coupled to a communication interface 412 such that server computer device 400 is capable of communicating with a remote device such as another server computer device 400, image server 102, and/or user device 104. For example, communication interface 412 may receive requests from user device 104 via the Internet.

Processor 405 may also be operatively coupled to a storage device 434. Storage device 434 may be any computer-operated hardware suitable for storing and/or retrieving data, such as, but not limited to, data associated with server system 108 (shown in FIG. 1). In some embodiments, storage device 434 may be integrated in server computer device 400. For example, server computer device 400 may include one or more hard disk drives as storage device 434.

In other embodiments, storage device 434 may be external to server computer device 400 and may be accessed by a plurality of server computer devices 400. For example, storage device 434 may include a storage area network (SAN), a network attached storage (NAS) system, and/or multiple storage units such as hard disks and/or solid-state disks in a redundant array of inexpensive disks (RAID) configuration.

In some embodiments, processor 405 may be operatively coupled to storage device 434 via a storage interface 420. Storage interface 420 may be any component capable of providing processor 405 with access to storage device 434. Storage interface 420 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 405 with access to storage device 434.

Processor 405 may execute computer-executable instructions for implementing aspects of the disclosure. In some embodiments, the processor 405 may be transformed into a special purpose microprocessor by executing computer-executable instructions or by otherwise being programmed. For example, the processor 405 may be programmed with the instructions such as illustrated in FIG. 2.

For the specification and the claims, reference will be made to a number of terms, which shall be defined to have the following meanings.

The singular forms “a,” “an,” and “the” include plural references unless the context clearly dictates otherwise.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

As used herein, the term “database” may refer to either a body of data, a relational database management system (RDBMS), or to both. As used herein, a database may include any collection of data including hierarchical databases, relational databases, flat file databases, object-relational databases, object oriented databases, and any other structured collection of records or data that is stored in a computer system. The above examples are example only, and thus are not intended to limit in any way the definition and/or meaning of the term database. Examples of RDBMS' include, but are not limited to including, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, California; IBM is a registered trademark of International Business Machines Corporation, Armonk, New York; Microsoft is a registered trademark of Microsoft Corporation, Redmond, Washington; and Sybase is a registered trademark of Sybase, Dublin, California.)

As used herein, the terms “processor” and “computer” and related terms, e.g., “processing device”, “computing device”, and “controller” are not limited to just those integrated circuits referred to in the art as a computer, but broadly refers to a microcontroller, a microcomputer, a programmable logic controller (PLC), an application specific integrated circuit (ASIC), and other programmable circuits, and these terms are used interchangeably herein. In the embodiments described herein, memory may include, but is not limited to, a computer-readable medium, such as a random-access memory (RAM), and a computer-readable non-volatile medium, such as flash memory. Alternatively, a floppy disk, a compact disc-read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

Further, as used herein, the terms “software” and “firmware” are interchangeable and include any computer program storage in memory for execution by personal computers, workstations, clients, servers, and respective processing elements thereof.

In another embodiment, a computer program is provided, and the program is embodied on a computer-readable medium. In an example embodiment, the system is executed on a single computer system, without requiring a connection to a server computer. In a further example embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device, and a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Furthermore, as used herein, the term “real-time” refers to at least one of the time of occurrence of the associated events, the time of measurement and collection of predetermined data, the time for a computing device (e.g., a processor) to process the data, and the time of a system response to the events and the environment. In the embodiments described herein, these activities and events may be considered to occur substantially instantaneously.

This written description uses examples to describe the disclosed embodiments, including the best mode, and also to enable any person skilled in the art to practice the disclosed embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A smart echocardiography (ECHO) system comprising:
a computer system comprising at least one processor in communication with at least one memory, wherein the at least one processor is programmed to:
access a two-stage machine-learning (ML) model for analyzing echocardiograms, wherein the two-stage ML model is trained to output a validated cardiac profile of a patient having improved accuracy based upon an inputted echocardiogram;
receive echocardiographic imaging data of a patient from the inputted echocardiogram;
execute a first-stage of the two-stage ML model to generate an initial cardiac profile based on the echocardiographic imaging data, the initial cardiac profile including a plurality of cardiac parameters each having a parameter value;
execute a second stage of the two-stage ML model on the initial cardiac profile including executing a plurality of validation calculations using the plurality of cardiac parameters and associated parameter values to generate a validated cardiac profile for the patient; and
output the validated cardiac profile.

2. The smart ECHO system of claim 1, wherein the parameter values of the plurality of cardiac parameters are determined based on at least one of image measurements from the echocardiographic imaging data and calculations performed based on the image measurements.

3. The smart ECHO system of claim 2, wherein the validated cardiac profile includes updated parameter values relative to the initial cardiac profile for at least one of the plurality of cardiac parameters.

4. The smart ECHO system of claim 2, wherein the plurality of cardiac parameters includes at least one of: i) blood flow direction along a sample line of flow, ii) blood flow velocity along the sample line of flow, iii) aortic and pulmonary valve stroke volume calculation, iv) mitral and tricuspid valve stroke volume calculation, v) shunt volume, and vi) transvalvular total net stroke volume.

5. The smart ECHO system of claim 1, wherein the at least one processor is programmed to determine a diagnosis for the patient based on the validated cardiac profile.

6. The smart ECHO system of claim 1, wherein the validated cardiac profile includes at least one of a structural, functional, and disease profile of a heart of the patient.

7. The smart ECHO system of claim 1, wherein the at least one processor is programmed to generate a report based on the validated cardiac profile.

8. The smart ECHO system of claim 1, wherein the two-stage ML model is trained based on historical echocardiogram data, the historical echocardiogram data including a plurality of historical echocardiographic imaging data and associated validated cardiac profiles.

9. The smart ECHO system of claim 1, wherein the at least one processor, based on the output validated cardiac profile, is programmed to perform a real-time dimensional analysis of blood flow direction and change in blood flow rate as a function of time.

10. The smart ECHO system of claim 1, wherein the plurality of cardiac parameters each represent at least one of a structural and functional feature of a heart of the patient and the parameter values represent a condition of the at least one of the structural and functional feature.

11. A non-transitory computer readable medium comprising instructions stored thereon, wherein the instructions, when executed by at least one processor, cause the at least one processor to:
access a two-stage machine-learning (ML) model for analyzing echocardiograms, wherein the two-stage ML model is trained to output a validated cardiac profile of a patient having improved accuracy based upon an inputted echocardiogram;
receive echocardiographic imaging data of a patient from the inputted echocardiogram;
execute a first-stage of the two-stage ML model to generate an initial cardiac profile based on the echocardiographic imaging data, the initial cardiac profile including a plurality of cardiac parameters each having a parameter value;
execute a second stage of the two-stage ML model on the initial cardiac profile including executing a plurality of validation calculations using the plurality of cardiac parameters and associated parameter values to generate a validated cardiac profile for the patient; and
output the validated cardiac profile.

12. The non-transitory computer readable medium of claim 11, wherein the parameter values of the plurality of cardiac parameters are determined based on at least one of image measurements from the echocardiographic imaging data and calculations performed based on the image measurements.

13. The non-transitory computer readable medium of claim 12, wherein the validated cardiac profile includes updated parameter values relative to the initial cardiac profile for at least one of the plurality of cardiac parameters.

14. The non-transitory computer readable medium of claim 12, wherein the plurality of cardiac parameters include at least one of: i) blood flow direction along a sample line of flow, ii) blood flow velocity along the sample line of flow, iii) aortic and pulmonary valve stroke volume calculation, iv) mitral and tricuspid valve stroke volume calculation, v) shunt volume, and vi) transvalvular total net stroke volume.

15. The non-transitory computer readable medium of claim 11, wherein the at least one processor is programmed to determine a diagnosis for the patient based on the validated cardiac profile.

16. The non-transitory computer readable medium of claim 11, wherein the validated cardiac profile includes at least one of a structural, functional, and disease profile of a heart of the patient.

17. The non-transitory computer readable medium of claim 11, wherein the two-stage ML model is trained based on historical echocardiogram data, the historical echocardiogram data including a plurality of historical echocardiographic imaging data and associated validated cardiac profiles.

18. A computer-implemented method for analyzing echocardiography (ECHO) results comprising:
accessing, from at least one memory, a two-stage machine-learning (ML) model for analyzing echocardiograms, wherein the two-stage ML model is trained to output a validated cardiac profile of a patient having improved accuracy based upon an inputted echocardiogram;
receiving echocardiographic imaging data of a patient from the inputted echocardiogram;
executing, by at least one processor in communication with the at least one memory, a first-stage of the two-stage ML model to generate an initial cardiac profile based on the echocardiographic imaging data, the initial cardiac profile including a plurality of cardiac parameters each having a parameter value;
executing, by the at least one processor, a second stage of the two-stage ML model on the initial cardiac profile including executing a plurality of validation calculations using the plurality of cardiac parameters and associated parameter values to generate a validated cardiac profile for the patient; and
outputting, by the at least one processor, the validated cardiac profile.

19. The method of claim 18, wherein the parameter values of the plurality of cardiac parameters are determined based on at least one of image measurements from the echocardiographic imaging data and calculations performed based on the image measurements.

20. The method of claim 19, wherein the validated cardiac profile includes updated parameter values relative to the initial cardiac profile for at least one of the plurality of cardiac parameters.
